(19) United States
(12) Patent Application Publication
(10) Pub. No.: US 2008/0194489 A1
(43) Pub. Date: Aug. 14, 2008

(54) Treatment of iatrogenic disease

(76) Inventors: Nisar A. Khan, Rotterdam (NL); Robbert Benner, Barendrecht (NL)

Correspondence Address: TRASK BRITT, P.O. BOX 2550, SALT LAKE CITY, UT, 84110, US

(21) Appl. No.: 11/981,491

(22) Filed: Oct. 30, 2007

Related U.S. Application Data
(63) Continuation-in-part of application No. 10/409,671, filed on Apr. 8, 2003.
Application No. 10/409,671 is a continuation-in-part of application No. 10/028,075, filed on Dec. 21, 2001.

(51) Int. Cl. A61K 38/07; A61K 38/08

(52) U.S. Cl. 514/16; 514/18; 514/17

(57) ABSTRACT

The invention relates to the field of human or veterinary medicine and to the treatment of subjects (be it man or animal) that suffer from iatrogenic disease, i.e., experience problems or complications resulting from a medical treatment. Provided is a method for modulating an iatrogenic event in a subject, the method comprising: providing the subject with a gene-regulatory peptide or functional analogue thereof. Furthermore, provided is the use of an NF-kappaB down-regulating peptide or functional analogue thereof for the production of a pharmaceutical composition for the treatment of an additional pro-inflammatory cytokine response occurring after an iatrogenic event in a subject.

### CROSS-REFERENCE TO RELATED APPLICATIONS

This application is a continuation-in-part of an earlier application U.S. patent application Ser. No. 10/409,671, filed Apr. 8, 2003, pending, which is a continuation-in-part of U.S. patent application Ser. No. 10/028,075, filed Dec. 21, 2001, pending, the contents of the entirety of each of which are hereby incorporated herein by this reference.

### TECHNICAL FIELD

The current invention relates to the body's innate way of modulation of important physiological processes and builds on insights reported in WO 99/59717, WO 01/00259 and PCT/NL/00639, the contents of the entirety of each are incorporated herein by this reference.

### BACKGROUND

U.S. Pat. No. 5,380,668 to Herron (Jan. 10, 1995), the contents of the entirety of which is incorporated by this reference, discloses, among other things, various compounds having the antigenic binding activity of hCG. Herron further discloses means and methods for making oligopeptides.

In these earlier applications small gene-regulatory peptides are described that are present naturally in pregnant women and are derived from proteolytic breakdown of placental gonadotropins such as human chorionic gonadotropin (hCG) produced during pregnancy. These peptides (in their active state often only at about four to six amino acids long) were shown to have unsurpassed immunological activity that they exert by regulating expression of genes encoding inflammatory mediators such as cytokines. Surprisingly, it was found that breakdown of hCG provides a cascade of peptides that help maintain a pregnant woman's immunological homeostasis. These peptides are nature's own substances that balance the immune system to assure that the mother stays immunologically sound while her fetus does not get prematurely rejected during pregnancy but instead is safely carried through its time of birth.

Where it was generally thought that the smallest breakdown products of proteins have no specific biological function on their own (except to serve as antigen for the immune system), it now emerges that the body in fact routinely utilizes the normal process of proteolytic breakdown of the proteins it produces to generate important gene-regulatory compounds, short peptides that control the expression of the body's own genes. Apparently, the body uses a gene-control system ruled by small broken down products of the exact proteins that are encoded by its own genes.

It is known that during pregnancy, the maternal system introduces a status of temporary immuno-modulation that results in suppression of maternal rejection responses directed against the fetus. Paradoxically, during pregnancy, often the mother's resistance to infection is increased and she is found to be better protected against the clinical symptoms of various auto-immune diseases such as rheumatism and multiple sclerosis. The protection of the fetus can thus not be interpreted only as a result of immune suppression. Each of the above three applications have provided insights by which the immunological balance between protection of the mother and protection of the fetus can be understood.

The inventors hereof have shown that certain short breakdown products of hCG (i.e., short peptides that can easily be synthesized, if needed modified, and used as pharmaceutical composition) exert a major regulatory activity on pro- or anti-inflammatory cytokine cascades that are governed by a family of crucial transcription factors, the NFkappaB family that stands central in regulating the expression of genes that shape the body's immune response.

Most of the hCG produced during pregnancy is produced by cells of the placenta, the exact organ where cells and tissues of mother and child most intensely meet and where immuno-modulation is most needed to fight off rejection. Being produced locally, the gene-regulatory peptides that are broken down from hCG in the placenta immediately balance the pro- or anti-inflammatory cytokine cascades found in the no-mans land between mother and child. Being produced by the typical placental cell, the trophoblast, the peptides traverse extracellular space; enter cells of the immune system and exert their immuno-modulatory activity by modulating NFkappaB-mediated expression of cytokine genes, thereby keeping the immunological responses in the placenta at bay.

### SUMMARY OF THE INVENTION

It is herein postulated that the beneficial effects seen on the occurrence and severity of auto-immune disease in the pregnant woman result from an overspill of the hCG-derived peptides into the body as a whole; however, these effects must not be overestimated, as it is easily understood that the further away from the placenta, the less immuno-modulatory activity aimed at preventing rejection of the fetus will be seen, if only because of a dilution of the placenta-produced peptides throughout the body as a whole. However, the immuno-modulatory and gene-regulatory activity of the peptides should by no means only be thought to occur during pregnancy and in the placenta; man and women alike produce hCG, for example, in their pituitaries, and nature certainly utilizes the gene-regulatory activities of peptides in a larger whole.

Consequently, a novel therapeutic inroad is provided, using the pharmaceutical potential of gene-regulatory peptides and derivatives thereof. Indeed, evidence of specific up- or down-regulation of NFkappaB driven pro- or anti-inflammatory cytokine cascades that are each, and in concert, directing the body's immune response was found in silico in gene-arrays by expression profiling studies, in vitro after treatment of immune cells and in vivo in experimental animals treated with gene-regulatory peptides. Also, considering that NFkappaB is a primary effector of disease (A. S. Baldwin, J. Clin. Invest., 2001, 107:3-6), using the hCG derived gene-regulatory peptides offer significant potential for the treatment of a variety of human and animal diseases, thereby tapping the pharmaceutical potential of the exact substances that help balance the mother's immune system such that her pregnancy is safely maintained.

### BRIEF DESCRIPTION OF THE DRAWINGS

FIG. 1: Hemorrhagic Shock model (HS) (* time of administration peptide A, B or C in the peptide groups).

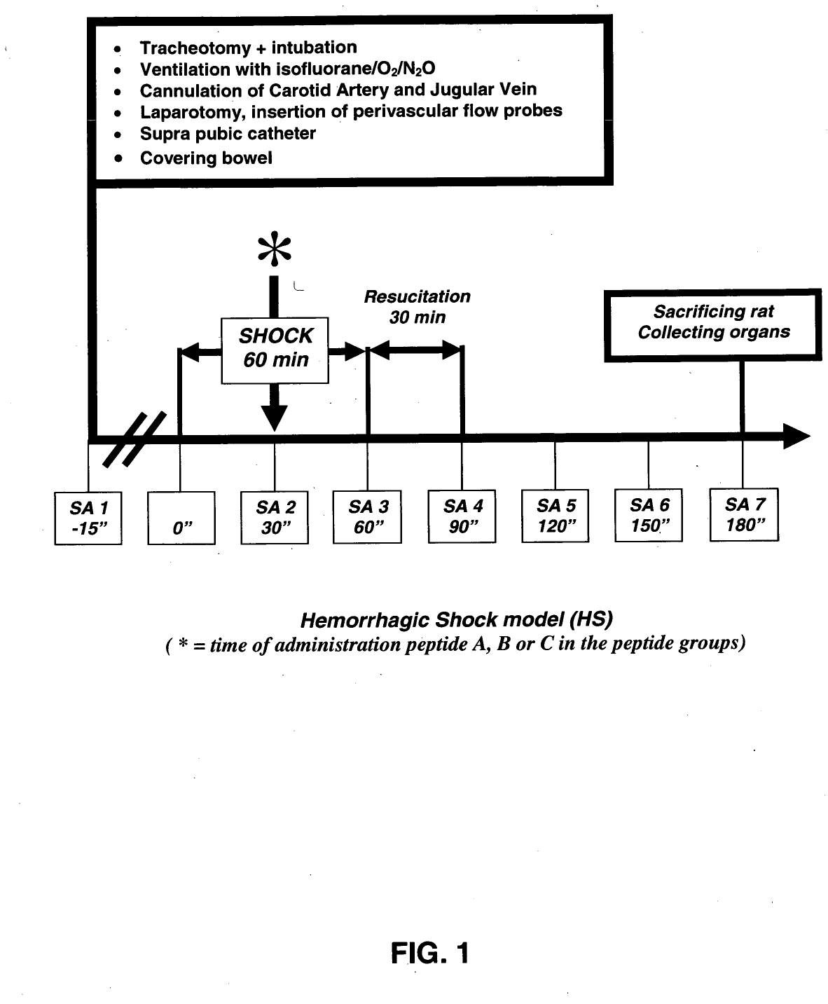

FIG. 2: Mean Arterial Pressure in sham, shock, and Peptide A, B and C experiments.

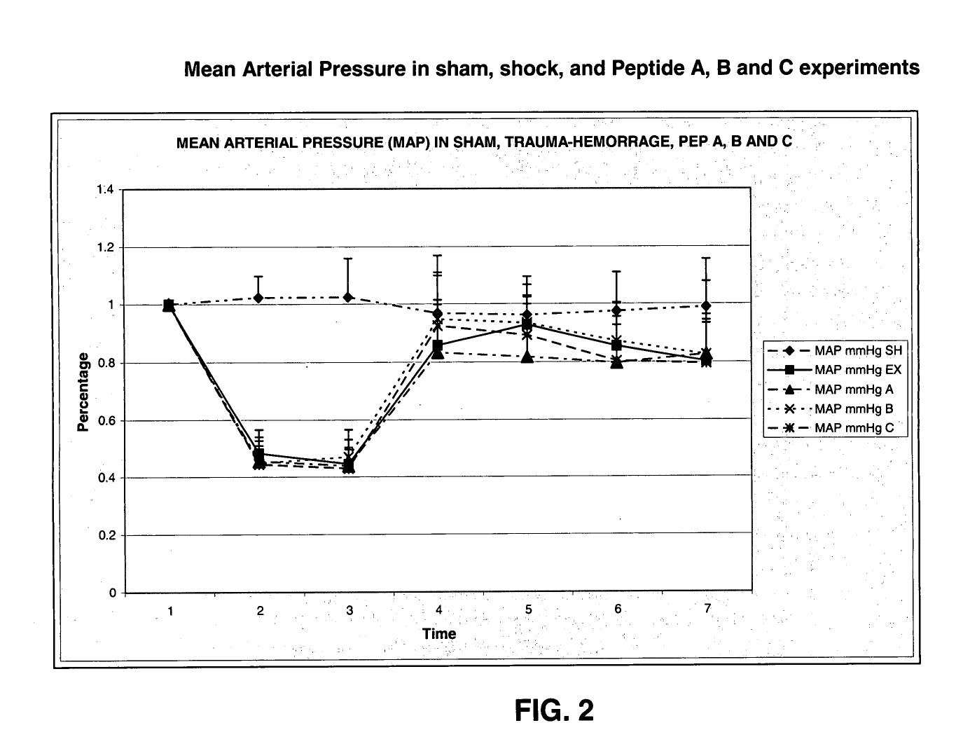

FIG. 3: Hematocrit in (from left to right) sham, shock, and Peptide A, B and C experiments.

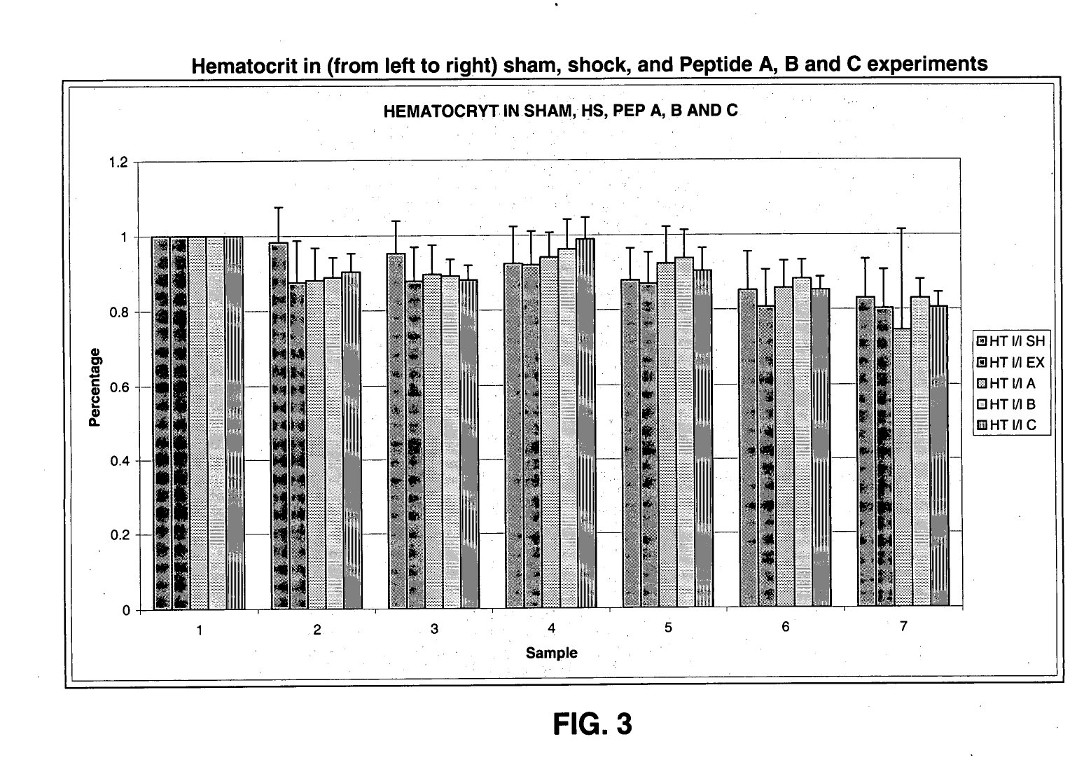

FIG. 4: Leukocytes during sham, trauma-hemorrhage, pep A, B and C experiments.

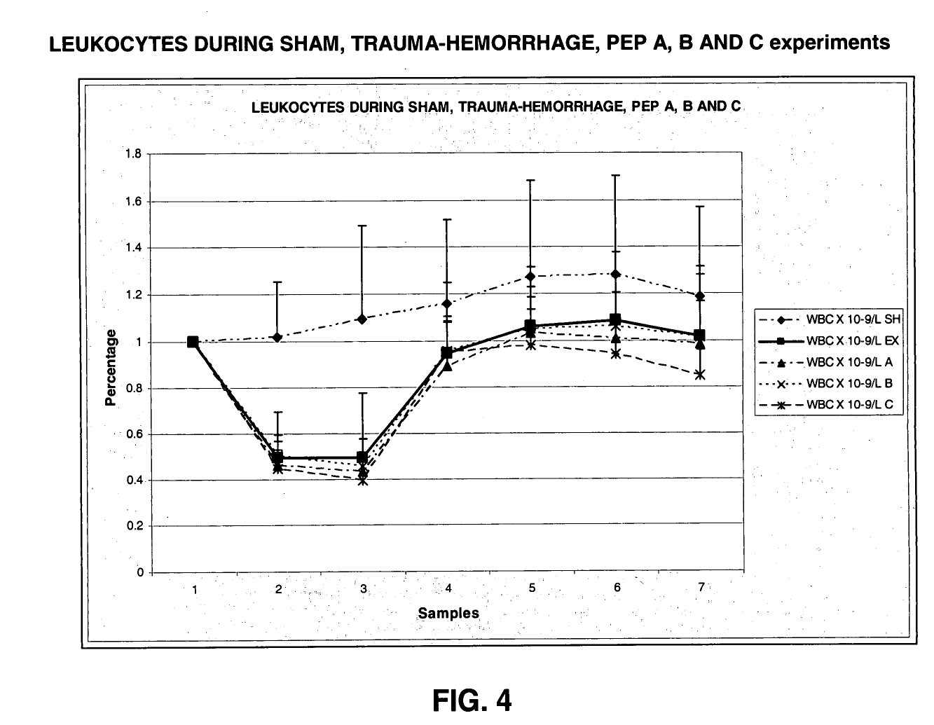

FIG. 5: Macrophages (MO) and granulocytes (GR) in (from left to right) sham, trauma-hemorrhagic shock, and Peptide A, B and C experiments.

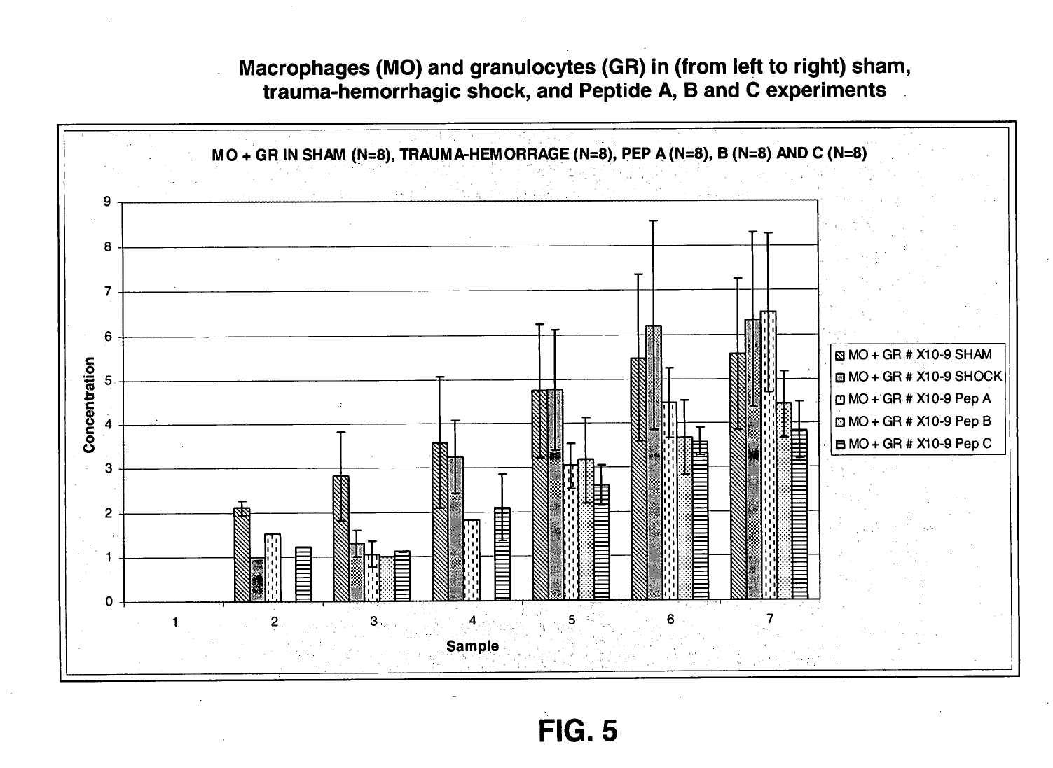

FIG. 6: Arterial blood flow in (from left to right) sham, shock, and Peptide A, B and C experiments.

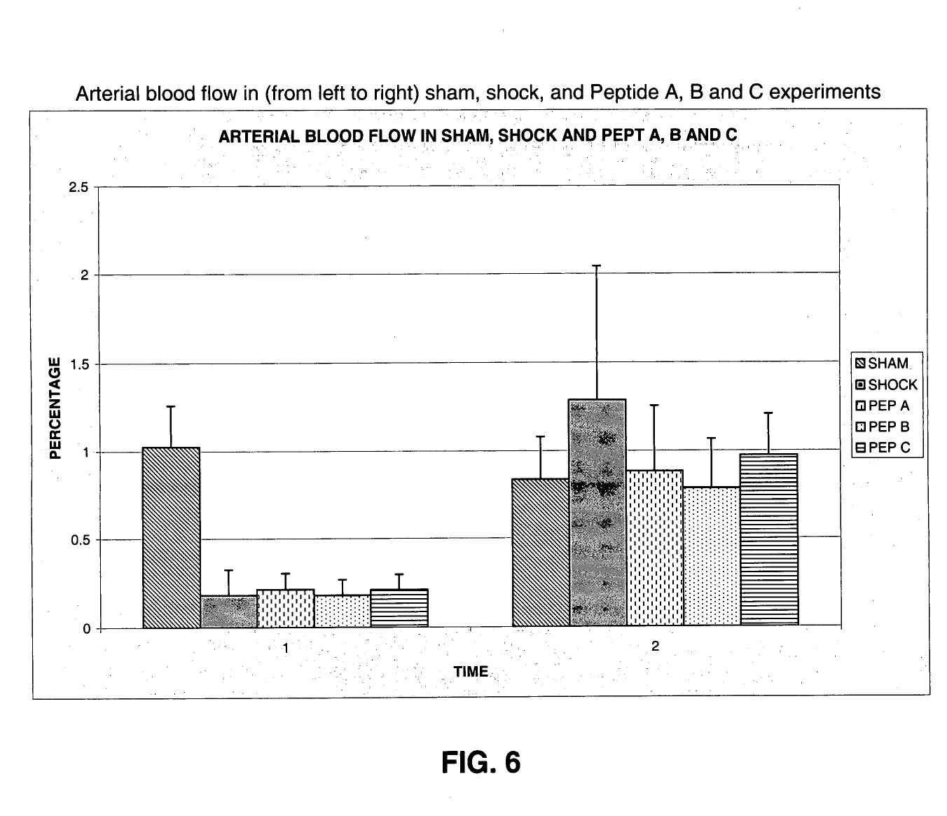

FIG. 7: Hemorrhagic shock model. A) Schematic representation of the experimental design. B) The measured mmHg was recalculated in percentages to standardize the experiment and to compensate for animal differences. C) Percentage of leukocytes in blood during various time points of the experiment.

FIG. 8: TNF-α plasma levels in different experimental groups determined at 15 minutes before and 30, 60, 90, 120, 150 and 180 minutes after the onset of hemorrhagic shock. □ Sham, ◯ HS, ∇ HS/LQGV, ⋄ HS/AQGV, Δ HS/LAGV. Each figure represents one animal.

FIG. 9: IL-6 plasma levels in different experimental groups determined at 120, 150 and 180 minutes after the onset of hemorrhagic shock □ Sham, ◯ HS, ∇ HS/LQGV, ⋄ HS/AQGV, Δ HS/LAGV. Each figure represents one animal.

FIG. 10: Transcript levels for A) TNF-α, B) IL-6 and C) ICAM-1 in the liver, 180 minutes after the onset of hemorrhagic shock. Data expressed are correlated to GAPDH expression. □ Sham, ◯ HS, ∇ HS/LQGV, ⋄ HS/AQGV, Δ HS/LAGV. Each figure represents one animal

FIGS. 11A and 11B: Depict the same experiment but reflect the scores of two independent observers RK and JV. Peptide A=LAGV; Peptide B=AQGV; Peptide G=VLPALPQ; Peptide I=LQGV. Treatment protocol: Daily application of 4% imiquimod cream (day 0-day 6) on shaved back; 300 μg/mouse peptide in PBS i.p. on days −1, 1, 3 and 5. Scoring for redness, scaling and skin thickness, daily, blindly. Cumulative score=redness+scaling+thickness (scale 0-12).

FIG. 12: Peptide I=LQGV. Treatment protocol: Daily application of 5% imiquimod cream (day 1-day 5) on shaved back and ear. Immediately before imiquimod application, treat skin from back and ear with petroleum ether to remove fat and scales (also groups not treated with petroleum ether); 500 μg/mouse peptide I in PBS i.p. on days 1, 3 and 5. Measuring ear thickness on days 1, 3, and 5. Scoring for redness, scaling and skin thickness, daily, blindly. Cumulative score=redness+scaling+thickness (scale 0-12).

### DETAILED DESCRIPTION OF THE INVENTION

The invention has application in the fields of veterinary or human medicine and to the treatment of subjects (be it man or animal) that suffer from iatrogenic disease, e.g., experience problems or complications resulting from a medical treatment.

Iatrogenic events that result from activities of, for example, physicians or surgeons are commonplace in modern medicine and can often not be avoided. Various adverse conditions can occur due to malpractice or neglect, such as wrongly selecting or executing a therapy, misplacing or forgetting to remove surgical utensils during surgery, and the like. However, most therapeutic or surgical interventions, even those well selected and properly executed, may, even beyond their beneficial effects, cause adverse conditions in a patient. Surgeons and clinicians employ various forms of therapy that, although regarded as essential for the patient's well-being, may in them selves have a detrimental effect on host defenses. Some of the morbidity (e.g., sepsis) seen in the post-operative period after major surgery may, in part, be due to inhibition of the immune response in the perioperative period, as a result of the anesthesia given and the nature of the surgery carried out.

Recently, a number of retrospective studies have suggested that blood transfusions may be immuno-inhibitory and detrimental to the survival of patients undergoing surgery for malignant disease. In patients with cancer, increased numbers of malignant cells enter the circulation during anesthesia and surgery; inhibition of anti-tumor host defenses during this phase may result in the enhanced metastatic dissemination of malignant cells and the establishment of occult tumor deposits. Studies in animals tend to substantiate such a hypothesis. Many of the drugs (e.g., chemotherapeutic agents) used to treat cancer and external beam radiotherapy, have well-documented and prolonged detrimental effects on aspects of the immune response. Recently, biological immune modulators (interferons, interleukins) have been introduced into clinical practice.

Recombinant technology is producing an ever-increasing variety of substances for potential use against cancer. Furthermore, also tried and tested therapies in infectious disease, such as treatments with antibiotics or antivirals, have their iatrogenic side-effects, often related to the lysis or destruction of the very micro-organism they are designed to be used against, and the release of microbe membrane fragments and/or toxins that induces additional pro-inflammatory cytokine release. For example, M. Norimatsu and D. C. Morrison (J. Infect. Dis. 1998 May, 177(5):1302-7), found a correlation of antibiotic-induced endotoxin release and cytokine production in Escherichia coli-inoculated mouse whole blood ex vivo. E. coli were incubated in mouse whole blood ex vivo supplemented with beta-lactam antibiotics that possessed preferential affinities for penicillin-binding proteins (PBPs). After four hours, viable bacteria were undetectable in the presence of any of the three antibiotics tested, whereas significant increases in colony-forming units were detected in samples not treated with antibiotics. Differential levels of endotoxin in platelet-rich plasma were detected using the limulus amebocyte lysate assay, according to differential antibiotic affinities for the various PBPs. Levels of tumor necrosis factor-alpha (TNF-alpha) and interleukin-6 (IL-6) in antibiotic-treated cultures after eight hours of incubation correlated well with the levels of endotoxin at four hours (r=0.96, P<0.0001 for TNF-alpha; r=0.91, P=0.0002 for IL-6). These data again indicate that antibiotics affect both endotoxin and cytokine responses and sometimes correlate negatively with in vivo protective efficacy of these antibiotics in gram-negative infections. Similar effects can be seen with the treatment of bacteraemia with so-called phage therapy, especially when a lytic phage is used.

Also, selective decontamination of the gut, as practiced in some patients, for example, in preparation of patient for a bone marrow transplantation, induces additional pro-inflammatory cytokine release, which can add to the pro-inflammatory burst in case of a complication such as hemorrhagic shock. Also, major surgery, such as cardiopulmonary bypass predisposes the splanchnic region to inadequate perfusion and increases in gut permeability. Related to these changes, circulating endotoxin has been shown to rise during surgery, and contributes to cytokine activation, high oxygen consumption, and fever (“post-perfusion syndrome”). To a large extent, free endotoxin in the gut is a product of the proliferation of aerobic gram-negative bacteria and may be reduced by nonabsorbable antibiotics, however, selective decontamination of the gut does not affect the occurrence of perioperative endotoxemia, nor does it reduce the tumor necrosis factor-alpha or interleukin-6 concentrations as determined before surgery, upon aorta declamping, 30 minutes into reperfusion, or two hours after surgery. Also, selective decontamination of the gut does not alter the incidence of postoperative fever or clinical outcome measures such as duration of artificial ventilation and intensive care unit and hospital stay. In conclusion, other measures are needed to affect the incidence of perioperative endotoxemia, pro-inflammatory cytokine activation and the occurrence of a post-perfusion syndrome during or after surgery.

Whatever the cause may be, most iatrogenic events, herein defined as a disorder or disease resulting from a treatment of a human or animal subject with a pharmaceutical composition or by a medical or surgical procedure, resulting in the damage, destruction or lysis of cells or tissue of the subject, resulting in additional pro-inflammatory cytokine release.

Typically, provided is a method for modulating such an iatrogenic event in a subject comprising providing the subject with a peptide selected from the group of LQG, AQG, LQGV (SEQ ID NO: 1 of the hereby incorporated accompanying sequence listing), AQGV (SEQ ID NO: 2), LQGA (SEQ ID NO: 3), VLPALP (SEQ ID NO: 4), ALPALP (SEQ ID NO: 5), VAPALP (SEQ ID NO: 6), ALPALPQ (SEQ ID NO: 7), VLPAAPQ (SEQ ID NO: 8), VLPALAQ (SEQ ID NO: 9), LAGV (SEQ ID NO: 10), VLAALP (SEQ ID NO: 11), VLPALA (SEQ ID NO: 12), VLPALPQ (SEQ ID NO: 13), VLAALPQ (SEQ ID NO: 14), VLPALPA (SEQ ID NO: 15), GVLPALP (SEQ ID NO: 16), LPGC (SEQ ID NO: 19), MTRV (SEQ ID NO: 20), MTR, and VVC. It is preferred that the peptide is selected from the group of LAGV, AQGV, VLPALPQ and LQGV, especially in case of reducing tissue specific local cytokine release. It is even more preferred that the peptide is selected from the group of LAGV (SEQ ID NO: 10), AQGV (SEQ ID NO: 2), and LQGV (SEQ ID NO: 1) in particular when the subject is at risk to experience a systemic inflammatory response syndrome occurring after the event, such as during or after surgery. Also provided is the use of these peptides when the iatrogenic event comprises destruction or lysis of a cell or tissue of the subject or of a pathogen hosted by the subject, for example, wherein the lysis is due to treatment of the subject with a pharmaceutical composition.

Provided is a pharmaceutical composition for the treatment of an iatrogenic event occurring in a subject, for example, in a primate, and a method for the treatment of an iatrogenic event resulting in additional pro-inflammatory cytokine release, for example, in a primate comprising subjecting the subject to a signaling molecule of the invention, preferably to a mixture of such signaling molecules. Administration of such a signaling molecule or mixture preferably occurs systemically, e.g., by intravenous or intraperitoneal administration and leads to a dampening of the effect of the additionally released pro-inflammatory cytokines. In a further embodiment, such treatment also comprises the use of, for example, an antimicrobial agent, however, especially when such use is otherwise contraindicated or at least considered at risk because of the chance of generating toxin loads that lead to an additional pro-inflammatory cytokine response because of lysis of the microbe subject to the action of those antibiotics in an individual thus treated.

In certain embodiments, provided is a method for modulating an iatrogenic event in a subject believed to be in need thereof comprising providing the subject with a signaling molecule comprising a short, gene regulatory peptide or functional analogue thereof, wherein the signaling molecule is administered in an amount sufficient to modulate the iatrogenic event. The signal molecule is preferably a short peptide, preferably of at most 30 amino acids long, or a functional analogue or derivative thereof.

In certain embodiments, the peptide is an oligopeptide of from about three to about fifteen amino acids long, preferably four to twelve, more preferably four to nine, most preferably four to six amino acids long, or a functional analogue or derivative thereof. Of course, such signaling molecule can be longer, for example, by extending it (N- and/or C-terminally), with more amino acids or other side groups, which can, for example, be (enzymatically) cleaved off when the molecule enters the place of final destination.

In particular, a method is provided wherein the signaling molecule modulates translocation and/or activity of a gene transcription factor. It is particularly useful when the gene transcription factor comprises an NF-kappaB/Rel protein or an AP-1 protein. Many of the iatrogenic events as mentioned above induce increased expression of inflammatory cytokines due to activation of NF-κB and/or AP-1, and in a preferred embodiment provided is a method wherein translocation and/or activity of the NF-kappaB/Rel protein or AP-1 protein is inhibited. In one embodiment, the peptide is selected from the group of peptides LQG, AQG, LQGV (SEQ ID NO:1), AQGV (SEQ ID NO:2), LQGA (SEQ ID NO:3), VLPALP (SEQ ID NO:4), ALPALP (SEQ ID NO:7), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLPALA (SEQ ID NO:12), VLPALPQ (SEQ ID NO:13), VLAALPQ (SEQ ID NO:14), VLPALPA (SEQ ID NO:14), GVLPALP (SEQ ID NO:16), LQGVLPALPQVVC (SEQ ID NO:17), LPGCPRGVNPVVS (SEQ ID NO:18), LPGC (SEQ ID NO:19), MTRV (SEQ ID NO:20), MTR, and VVC. As said, additional expression of inflammatory cytokines is often due to activation of NF-κB and AP-1. Inflammatory cytokines can be expressed by endothelium (for example, by trauma), perivascular cells and adherent or transmigrating leukocytes, inducing numerous pro-inflammatory and procoagulant effects. Together these effects predispose to inflammation, thrombosis and hemorrhage.

Of clinical and medical interest and value is the opportunity to selectively control NFκB-dependent gene expression in tissues and organs in a living subject, preferably in a primate, allowing up-regulating essentially anti-inflammatory responses such as IL-10, and down-regulating essentially pro-inflammatory responses such as mediated by TNF-alpha, nitric oxide (NO), IL-5, IL-6 and IL-1beta.

Provided is use of a NFκB regulating peptide or derivative thereof for the production of a pharmaceutical composition for the treatment of an iatrogenic event, preferably in a primate, and provides a method of treatment of an iatrogenic event, notably in a primate. It is preferred when the treatment comprises administering to the subject a pharmaceutical composition comprising an NFkappaB down-regulating peptide or functional analogue thereof. Examples of useful NFkappaB down-regulating peptides are VLPALPQVVC (SEQ ID NO:_), LQGVLPALPQ (SEQ ID NO:_), LQG, LQGV (SEQ ID NO:1), GVLPALPQ, VLPALP (SEQ ID NO:4), VVC, MTR and circular LQGVLPALPQVVC (SEQ ID NO:17). More down-regulating peptides and functional analogues can be found using the methods as provided herein. Most prominent among NFkappaB down-regulating peptides are VLPALPQVVC (SEQ ID NO:_), LQGVLPALPQ (SEQ ID NO:_), LQG, LQGV (SEQ ID NO:1, and VLPALP (SEQ ID NO:4). These are also capable of reducing production of NO by a cell. It is herein also provided to use a composition that comprises at least two oligopeptides or functional analogues thereof, each capable of down-regulating NFkappaB, and thereby reducing production of NO and/or TNF-alpha by a cell, in particular wherein the at least two oligopeptides are selected from the group LQGV (SEQ ID NO:1), AQGV (SEQ ID NO:2), and VLPALP (SEQ ID NO:4), for the treatment of an iatrogenic event.

In another embodiment, provided is a mode of treatment of the additional pro-inflammatory cytokine response seen after surgical interventions. Also, selective decontamination of the gut, as practiced in some patients, for example, in preparation of the patient for a bone marrow transplant, induces additional proinflammatory cytokine release, which can add to the proinflammatory burst in case of a complication such as hemorrhagic shock.

In another embodiment, provided is a method for clinical intervention wherein the iatrogenic event comprises destruction or lysis of a cell or tissue of the subject or of a pathogen hosted by the subject, for example, wherein the lysis is due to treatment of the subject with a pharmaceutical composition. Treatment of the invention can be achieved concomitantly with classical treatment. In response to a variety of signals received by the body as a consequence of the iatrogenic event, the NFkB/Rel family of transcription factors are activated and form different types of hetero- and homodimers among themselves to regulate the expression of target genes containing kappaB-specific binding sites. NF-kB transcription factors are hetero- or homodimers of a family of related proteins characterized by the Rel homology domain. They form two subfamilies, those containing activation domains (p65-RELA, RELB, and c-REL) and those lacking activation domains (p50, p52). The prototypical NFkB is a heterodimer of p65 (RELA) and p50 (NF-kB1). Among the activated NFkB dimers, p50-p65 heterodimers are known to be involved in enhancing the transcription of target genes and p50-p50 homodimers in transcriptional repression. However, p65-p65 homodimers are known for both transcriptional activation and repressive activity against target genes. KappaB DNA binding sites with varied affinities to different NFB dimers have been discovered in the promoters of several eukaryotic genes and the balance between activated NFkB homo- and heterodimers ultimately determines the nature and level of gene expression within the cell.

The term “NFkB-regulating peptide” as used herein refers to a peptide or a modification or derivative thereof capable of modulating the activation of members of the NFkB/Rel family of transcription factors. Activation of NFkB can lead to enhanced transcription of target genes. Also, it can lead to transcriptional repression of target genes. NFkB activation can be regulated at multiple levels. For example, the dynamic shuttling of the inactive NFkB dimers between the cytoplasm and nucleus by IkappaB proteins and its termination by phosphorylation and proteasomal degradation, direct phosphorylation, acetylation of NFkB factors, and dynamic reorganization of NFkB subunits among the activated NFkB dimers have all been identified as key regulatory steps in NFkB activation and, consequently, in NFkB-mediated transcription processes. Thus, an NFkB-regulating peptide is capable of modulating the transcription of pro-inflammatory cytokine genes that are under the control of NFkB/Rel family of transcription factors. Modulating comprises the up-regulation or the down-regulation of transcription.

In certain embodiments, a peptide of the invention, or a functional derivative or analogue thereof is used for the production of a pharmaceutical composition for the treatment of iatrogenic events. NFkappaB regulating peptide can be given concomittantly, the peptide (or analogue) concentration preferably being from about 1 to about 1000 mg/l, but the peptide can also been given in a bolus injection. Doses of 1 to 5 mg/kg bodyweight, for example, every eight hours in a bolus injection or per infusionem until the patient stabilizes, are recommended. For example, in cases where large adverse response to the iatrogenic event are expected or diagnosed, it is preferred to monitor cytokine profiles and arachidonic acid metabolites, such as, TNF-alpha, IL-6 or IL-10, and PGE2 and leukotrien levels, in the plasma of the treated patient, and to stop treatment of the invention when these levels are normal.

In certain embodiments, provided is a method of treating a subject suffering from an iatrogenic event with a method and signaling molecule of the invention concomitantly, or at least timely, with a thrombolytic agent, such as (recombinant) tissue plasminogen activator, or truncated forms thereof having tissue plasminogen activity, or streptokinase, or urokinase.

Also provided is a method for modulating an iatrogenic event in a subject that is concomitantly or already treated with a pharmaceutical composition and believed to be suffering or at risk from the side-effects of such a composition comprising providing the subject with a signaling molecule comprising a short, gene regulatory peptide or functional analogue thereof, wherein the signaling molecule is administered in an amount sufficient to modulate the side-effects, for example, wherein the pharmaceutical composition is selected from the group of antigens, vaccines, antibodies, anticoagulants, antibiotics, in particular beta-lactam antibiotics, antitoxins, antibacterial agents, antiparasitic agents, antiprotozootic agents, antifungal agents, antiviral agents, cytolytic agents, cytostatic agents, thrombolytic agents, and others. The invention, for example, provides a method to control the toxic effects of the lysis or damage to bacterial pathogens that release endotoxin and a host of other enterotoxin and exotoxins, resulting in at times an undesirable pro-inflammatory cytokine cascade.

As shown herein, the concept that controlling bacterial infections with antibiotics or lytic phages may control disease is flawed with the effect of antibiotics or phages that may kill the circulating or fixed pathogens but then at the same time release the toxins inherent to the bacterial walls or cytoplastic milieu. These antigens can induce multisystem organ disease or MODS and can trigger and be the source for the development of ARDS, SIRS, and DIC. This combines the effect of the chemokine or migratory system and the cytokine proinflammatory system that increases permeability and leakage of vascular elements and is toxic to many organ systems. Treating the patient with an NFkappaB down-regulating peptide as well mitigates these effects. For this purpose, it is herein provided to provide the patient with a bolus injection of NF-kappaB down-regulating peptide such as AQGV, LQGV or VLPALP at 2 mg/kg and continue an infusion with an NF-kappaB down-regulating peptide such as AQGV, LQGV or VLPALP or a functional analogue thereof at a dose of 1 mg/kg bodyweight for every eight hours. Dosages may be increased or decreased, for example, depending on the outcome of monitoring the cytokine profile in the plasma of the patient.

In certain embodiments, a signal molecule is administered in an effective concentration to an animal or human systemically, e.g., by intravenous, intra-muscular or intraperitoneal administration. Another way of administration comprises perfusion of organs or tissue, be it in vivo or ex vivo, with a perfusion fluid comprising a signal molecule of the invention. Bacteriophages act by killing or inactivating bacteria in a different way than most antibiotics that may already have some protective elements, which lessen the likelihood of septic shock and all of its elements. Phage may change the apoptotic pathways that clear such dying or dead pathogens in such a way that these organisms are sequestered from releasing their toxins to the host. This differs dramatically from the penicillins and cephalosporin families of antibiotics that effect the destruction of bacterial cell walls or drugs such as aminoglycosides that effect protein synthesis within the bacterial cells.

The penicillins are bactericidal antibiotics that impair synthesis of the bacterial cell wall peptidoglycan by attaching to penicillin-binding proteins located on the inner surface of the cell membrane. At least eight penicillin-binding proteins have been identified; they are the enzymes that are responsible for linking individual elements of the bacterial cell wall together. The cephalosporins and closely related cephamycins (e.g., cefoxitin and cefotetan) are a large and rapidly expanding group of beta-lactam antibiotics. Like the penicillins, the cephalosporins are bactericidal antibiotics that inhibit bacterial cell wall synthesis and have a low intrinsic toxicity. The adverse effects of the cephalosporins are mainly hypersensitivity reactions, local pain (with intramuscular use), and thrombophlebitis (with intravenous use). Less common toxicities include GI symptoms, elevated liver enzyme levels, and renal impairment; third- and fourth-generation cephalosporins may cause seizures, including nonconvulsive status epilepticus, in patients with renal failure. Imipenem and meropenem were the first carbapenems available for clinical use in the United States; the third, ertapenem, was released in 2002. Like other beta-lactam antibiotics, they are bactericidal and act by inhibiting bacterial cell wall synthesis.

Three properties account for the extraordinarily broad antibacterial spectrum of the carbapenems: there is no permeability barrier excluding the drugs from bacteria; they have high affinity for penicillin-binding protein 2 (PBP-2), which is a crucial component of cell wall structure; and they are extremely resistant to hydrolysis by beta-lactamases. The aminoglycosides are bacterial drugs that act by binding irreversibly to the 30S ribosomal subunit of susceptible bacteria. Because oxygen is required to transport aminoglycosides across the outer bacterial membrane, these agents are ineffective against anaerobes and may function poorly in the anaerobic milieu of abscesses.

Although various aminoglycosides display activity against a wide range of microorganisms, they are used chiefly to treat infections caused by aerobic gram-negative bacilli. Aminoglycosides are also used in combination with cell wall-active antibiotics (e.g., penicillins and vancomycin) for the synergistic treatment of deep tissue infections caused by enterococci and coagulase-negative staphylococci. In addition, streptomycin is still used to treat tuberculosis, tularemia, and plague. Vancomycin is a bactericidal glycopeptide that impairs cell wall synthesis of gram-positive bacteria; its spectrum of action includes staphylococci, streptococci, pneumococci, enterococci, clostridia, Corynebacterium species, and other gram-positive bacteria. It is bacteriostatic but not bactericidal against some strains of enterococci, coagulase-negative staphylococci, and corynebacteria. Quinupristin and dalfopristin are two structurally distinct streptogramins that bind to separate sites on the bacterial 50S ribosomal subunit; they thus act synergistically to inhibit protein synthesis.

Although quinupristin-dalfopristin is active against a variety of bacteria, its major use is in the treatment of serious infections caused by vancomycin-resistant strains of E. faecium. The drugs may also be useful in occasional vancomycin-intolerant patients with severe infections caused by methicillin-resistant S. aureus or coagulase-negative staphylococci. Although quinupristin-dalfopristin was first marketed in 1999, resistance is already emerging. In 2000, linezolid became the first member of the oxazolidinone class to be approved for clinical use in the United States. Linezolid is a synthetic antibiotic that inhibits protein synthesis by binding to a site on the bacterial 23S ribosomal RNA of the 50S subunit, thus preventing function of the initiation complex that is required for ribosomal function. Because no other antibiotic acts in this way, bacteria that have developed resistance to other ribosomally active antimicrobials do not display cross-resistance to linezolid. Linezolid is active against nearly all aerobic gram-positive cocci at concentrations of 4 mg/ml or less, including penicillin-resistant pneumococci, methicillin-resistant staphylococci, and vancomycin-resistant enterococci; however, resistant strains have been isolated. The drug is bacteriostatic against staphylococci and enterococci, but it is bactericidal against most streptococcal strains. Linezolid is also active against L. monocytogenes, M. catarrhalis, H. influenzae, N. gonorrhoeae, B. pertussis, Pasteurella multocida, and Nocardia species. C. difficile, C. perfringens, and Bacteroides species are susceptible, but enteric gram-negative bacilli and Pseudomonas species are not. Metronidazole was first approved in 1959 for use as an antiparasitic agent; in 1981, the FDA approved an intravenous preparation of metronidazole for the treatment of serious infections caused by anaerobic bacteria. Orally administered metronidazole is excellent for treating pseudomembranous colitis caused by C. difficile. Metronidazole is also useful as part of preoperative prophylactic regimens for elective colorectal surgery. The drug is being studied for use in the treatment of nonspecific vaginitis, which is associated with Gardnerella vaginalis. Metronidazole appears to act by disrupting bacterial DNA and inhibiting nucleic acid synthesis. The drug is bactericidal against almost all anaerobic gram-negative bacilli, including B. fragilis, and against most Clostridium species. Although true anaerobic streptococci are generally susceptible to metronidazole, microaerophilic streptococci and Actinomyces and Propionibacterium species are often resistant. Metronidazole has cured a variety of infections caused by anaerobes: CNS infections, bone and joint infections, abdominal and pelvic sepsis, and endocarditis. Failures have been reported in the treatment of pleuropulmonary infections.

The addition of a fluorine group and a piperazine substituent to the first quinolones has greatly improved the antibacterial spectrum of this class of drugs; the addition of a methyl group on the piperazine ring appears to further enhance the bioavailability of these compounds. There are currently ten quinolones available for clinical use in the United States, and many additional fluoroquinolones are actively being studied. The fluoroquinolones are bactericidal compounds that act by inhibiting DNA gyrase, the bacterial enzyme responsible for maintaining the supertwisted helical structure of DNA; DNA topoisomerase IV is a secondary target. The fluoroquinolones rapidly kill bacteria, probably by impairing DNA synthesis and possibly by mechanisms involving cleaving of bacterial chromosomal DNA. Bacterial resistance to the fluoroquinolones depends on a change in their DNA gyrase. In the case of nalidixic acid, this single-step mutation occurs with a frequency of 10⁻⁷; resistance to the newer fluoroquinolones occurs much less frequently (about 10⁻¹¹) but is a growing concern.

Bacterial strains that are resistant to one fluoroquinolone tend to be cross-resistant to related compounds; such resistance is usually mediated by chromosomes, but plasmid-mediated resistance raises the possibility of transferable resistance. The fluoroquinolones are broad-spectrum antimicrobials. Most enteric gram-negative bacilli, including E. coli, Proteus, Klebsiella, and Enterobacter, are highly susceptible; common GI pathogens, such as Salmonella, Shigella, and Campylobacter species, are also very sensitive. Other gram-negative organisms that are killed by low concentrations of the fluoroquinolones are N. gonorrhoeae, N. meningitidis, H. influenzae, P. multocida, M. catarrhalis, and Y. enterocolitica. Acinetobacter and Serratia are somewhat less susceptible. P. aeruginosa is susceptible to ciprofloxacin and trovafloxacin; ofloxacin and levofloxacin are moderately active, but the other quinolones are not effective. P. cepacia and S. maltophila are quinolone-resistant. Ciprofloxacin is the drug of choice for B. anthracis; oflaxacin and levofloxacin are also active in vitro. Among gram-positive cocci, methicillin-sensitive strains of S. aureus and coagulase-negative staphylococci are usually susceptible to quinolones, but methicillin-resistant S. aureus and enterococci are not. Lomefloxacin is not active against pneumococci and other streptococci; ciprofloxacin and ofloxacin are moderately active; and levofloxacin, sparfloxacin, gatifloxacin, moxifloxacin, and trovafloxacin are highly effective, even against non-penicillin-sensitive pneumococci.

Even fastidious intracellular pathogens can be inhibited by the quinolones; Chlamydia, Mycoplasma, Listeria, Legionella, and M. tuberculosis are in this category. Only trovafloxacin is highly active against anaerobes. Levofloxacin, gatifloxacin, moxifloxacin, and sparfloxacin demonstrate some activity against anaerobes, but the other quinolones do not. C. difficile is resistant to quinolones.

Many of these families of antibiotics are walking a tightrope into the future because of resistant bacteria that can transfer their plasmids, or can be effective through the strength of their efflux pumps or their protected environments of being part of a biofilm. Both gram positive and gram negative bacteria have toxins and enzymes that cause pathogenesis. Other pathogens such as fungi and specific parasites also have these complex toxins and glycoproteins that can also mimic bacterial septic shock or an apoptotic and DIC like reaction. Most pathogens induce degrees of cytokine inflammatory response that attempt to isolate the, for example, bacteria and eventually kill them but at the same time often lead to the very damage that is the hallmark of the infectious illness. This is akin to friendly fire in a war.

The use of a NFkappaB down-regulating peptide in conjunction with any of the above-mentioned antibiotics or in conjunction with lytic phage or even as a cocktail for concomitant use will prevent the ultimate damage unleashed by the primary infection first and then the secondary debris and release of toxins through mediation of the action of killing these bacteria with antibiotic or phage or coordination of both. Herewith, provided is a clinical approach where we can salvage the host even late into the disease process. This use of a peptide or functional analogue of the invention in conjunction with antibiotics or phage turns the odds of survival of the host into a highly likely occurrence and further diminishes the end-stage morbidity seen with these pathogens.

Also provided is a method wherein the iatrogenic event includes the treatment of a subject with a virus, especially wherein lysis is due to treatment of the subject with the virus. A clear example of the beneficial use of a peptide or functional analogue thereof of the invention to control a therapy-impeding inflammatory reaction relates to the example of an inflammatory response to (for example, adenoviral or retroviral) gene vectors, e.g., in gene therapy such as in treatment of cystic fibrosis. The peptides can be administered systemically as indicated above in the case of cystic fibrosis gene therapy. In another example, the virus comprises a lytic phage used in antibacterial therapy as discussed above.

The compounds according to the general formula may be prepared in a manner conventional for such compounds. To that end, suitably N alpha protected (and side-chain protected if reactive side-chains are present) amino acid derivatives or peptides are activated and coupled to suitably carboxyl protected amino acid or peptide derivatives either in solution or on a solid support. Protection of the alpha-amino functions generally takes place by urethane functions such as the acid-labile tertiary-butyloxycarbonyl group (“Boc”), benzyloxycarbonyl (“Z”) group and substituted analogs or the base-labile 9-fluoremyl-methyloxycarbonyl (“Fmoc”) group. The Z group can also be removed by catalytic hydrogenation. Other suitable protecting groups include the Nps, Bmv, Bpoc, Aloc, MSC, etc. A good overview of amino protecting groups is given in The peptides, Analysis, Synthesis, Biology, Vol. 3, E. Gross and J. Meienhofer, eds. (Academic Press, New York, 1981).

Protection of carboxyl groups can take place by ester formation, for example, base-labile esters like methyl or ethyl, acid labile esters like tert. butyl or, substituted, benzyl esters or hydrogenolytically. Protection of side-chain functions like those of lysine and glutamic or aspartic acid can take place using the aforementioned groups. Protection of thiol, and although not always required, of guanidino, alcohol and imidazole groups can take place using a variety of reagents such as those described in The Peptides, Analysis, Synthesis, Biology, id., or in Pure and Applied Chemistry, 59(3), 331-344 (1987). Activation of the carboxyl group of the suitably protected amino acids or peptides can take place by the azide, mixed anhydride, active ester, or carbodiimide method especially with the addition of catalytic and racemization-suppressing compounds like 1-N-N-hydroxybenzotriazole, N-hydroxysuccinimide, 3-hydroxy-4-oxo-3,4-dihydro-1,2,3,-benzotria-zine, N-hydroxy-5 norbornene-2,3-dicar-boxyimide. Also, the anhydrides of phosphorus-based acids can be used. See, e.g., The Peptides, Analysis, Synthesis, Biology, supra and Pure and Applied Chemistry, 59(3), 331-344 (1987).

It is also possible to prepare the compounds by the solid phase method of Merrifield. Different solid supports and different strategies are known, see, e.g., Barany and Merrifield in The Peptides, Analysis, Synthesis, Biology, Vol. 2, E. Gross and J. Meienhofer, eds. (Acad. Press, New York, 1980); Kneib-Cordonier and Mullen in Int. J. Peptide Protein Res., 30, 705-739 (1987); and Fields and Noble in Int. J. Peptide Protein Res., 35, 161-214 (1990). The synthesis of compounds in which a peptide bond is replaced by an isostere, can, in general, be performed using the previously described protecting groups and activation procedures. Procedures to synthesize the modified isosteres are described in the literature, for instance, for the —CH₂—NH— isostere and for the —CO—CH₂— isostere.

Removal of the protecting groups, and, in the case of solid phase peptide synthesis, the cleavage from the solid support, can take place in different ways, depending on the nature of those protecting groups and the type of linker to the solid support. Usually deprotection takes place under acidic conditions and in the presence of scavengers. See, e.g., volumes 3, 5 and 9 of the series on The Peptides Analysis, Synthesis, Biology, supra.

Another possibility is the application of enzymes in synthesis of such compounds; for reviews see, e.g., H. D. Jakubke in The Peptides, Analysis, Synthesis, Biology, Vol. 9, S. Udenfriend and J. Meienhofer, eds. (Acad. Press, New York, 1987).

Although possibly not desirable from an economic point of view, oligopeptides of the invention could also be made according to recombinant DNA methods. Such methods involve the preparation of the desired oligopeptide thereof by means of expressing recombinant polynucleotide sequence that codes for one or more of the oligopeptides in question in a suitable microorganism as host. Generally the process involves introducing into a cloning vehicle (e.g., a plasmid, phage DNA, or other DNA sequence able to replicate in a host cell) a DNA sequence coding for the particular oligopeptide or oligopeptides, introducing the cloning vehicle into a suitable eukaryotic or prokaryotic host cell, and culturing the host cell thus transformed. When a eukaryotic host cell is used, the compound may include a glycoprotein portion.

As used herein, a “functional analogue” or “derivative” of a peptide includes an amino acid sequence, or other sequence monomers, which has been altered such that the functional properties of the sequence are essentially the same in kind, not necessarily in amount. An analogue or derivative can be provided in many ways, for instance, through “conservative amino acid substitution.” Also peptidomimetic compounds can be designed that functionally or structurally resemble the original peptide taken as the starting point but that are, for example, composed of non-naturally occurring amino acids or polyamides. With “conservative amino acid substitution,” one amino acid residue is substituted with another residue with generally similar properties (size, hydrophobicity), such that the overall functioning is likely not to be seriously affected. However, it is often much more desirable to improve a specific function. A derivative can also be provided by systematically improving at least one desired property of an amino acid sequence. This can, for instance, be done by an Ala-scan and/or replacement net mapping method. With these methods, many different peptides are generated, based on an original amino acid sequence but each containing a substitution of at least one amino acid residue. The amino acid residue may either be replaced by alanine (Ala-scan) or by any other amino acid residue (replacement net mapping). This way, many positional variants of the original amino acid sequence are synthesized. Every positional variant is screened for a specific activity. The generated data are used to design improved peptide derivatives of a certain amino acid sequence.

A derivative or analogue can also be, for instance, generated by substitution of an L-amino acid residue with a D-amino acid residue. This substitution, leading to a peptide that does not naturally occur in nature, can improve a property of an amino acid sequence. It is, for example, useful to provide a peptide sequence of known activity of all D-amino acids in retro inversion format, thereby allowing for retained activity and increased half-life values. By generating many positional variants of an original amino acid sequence and screening for a specific activity, improved peptide derivatives comprising such D-amino acids can be designed with further improved characteristics.

A person skilled in the art is well able to generate analogous compounds of an amino acid sequence. This can, for instance, be done through screening of a peptide library. Such an analogue has essentially the same functional properties of the sequence in kind, not necessarily in amount. Also, peptides or analogues can be circularized, for example, by providing them with (terminal) cysteines, dimerized or multimerized, for example, by linkage to lysine or cysteine or other compounds with side-chains that allow linkage or multimerization, brought in tandem- or repeat-configuration, conjugated or otherwise linked to carriers known in the art, if only by a labile link that allows dissociation.

As used herein, an oligopeptide also includes, for example, an acceptable salt, base, or ester of the oligopeptide or a labeled oligopeptide. As used herein, “acceptable salt” refers to salts that retain the desired activity of the oligopeptide or equivalent compound, but preferably do not detrimentally affect the activity of the oligopeptide or other component of a system in which uses the oligopeptide. Examples of such salts are acid addition salts formed with inorganic acids, for example, hydrochloric acid, hydrobromic acid, sulfuric acid, phosphoric acid, nitric acid, and the like. Salts may also be formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, and the like. Salts may be formed with polyvalent metal cations such as zinc, calcium, bismuth, barium, magnesium, aluminum, copper, cobalt, nickel and the like or with an organic cation formed from N,N′-dibenzylethylenediamine or ethylenediamine, or combinations thereof (e.g., a zinc tannate salt).

The oligopeptide, or its modification or derivative, can be administered as the entity, as such, or as a pharmaceutically acceptable acid- or base addition salt, formed by reaction with an inorganic acid (such as hydrochloric acid, hydrobromic acid, perchloric acid, nitric acid, thiocyanic acid, sulfuric acid, and phosphoric acid); or with an organic acid (such as formic acid, acetic acid, propionic acid, glycolic acid, lactic acid, pyruvic acid, oxalic acid, malonic acid, succinic acid, maleic acid, and fumaric acid); or by reaction with an inorganic base (such as sodium hydroxide, ammonium hydroxide, potassium hydroxide); or with an organic base (such as mono-, di-, trialkyl and aryl amines and substituted ethanolamines). A selected peptide and any of the derived entities may also be conjugated to sugars, lipids, other polypeptides, nucleic acids and PNA; and function in-situ as a conjugate or be released locally after reaching a targeted tissue or organ.

A pharmaceutical composition for use herein may be administered to the subject parenterally or orally. Such a pharmaceutical composition may consist essentially of (or consist of) oligopeptide and PBS. It is preferred that the oligopeptide is of synthetic origin. Suitable treatment, for example, entails administering the oligopeptide (or salt or ester) in the pharmaceutical composition to the patient intravenously in an amount of from about 0.0001 to about 35 mg/kg body mass of the subject. It may be useful that the pharmaceutical composition consists essentially of from one to three different oligopeptides.

In certain embodiments, a peptide of the invention, or a functional derivative or analogue thereof is used for the production of a pharmaceutical composition, for the treatment or mitigation of the additional pro-inflammatory cytokine response seen after an iatrogenic event. Examples of useful NFkappaB down-regulating peptides to be included in such a pharmaceutical composition are VLPALPQVVC (SEQ ID NO:_), LQGVLPALPQ, LQG (SEQ ID NO:_), LQGV (SEQ ID NO:1), GVLPALPQ (SEQ ID NO:_), VLPALP (SEQ ID NO:4), VVC, MTR and circular LQGVLPALPQVVC (SEQ ID NO:_). More gene-regulating peptides and functional analogues can be found in a (bio)assay, such as an NFkappaB translocation assay as provided herein. Most prominent among NFkappaB down-regulating peptides are VLPALPQVVC (SEQ ID NO:_), LQGVLPALPQ (SEQ ID NO:_), LQG, LQGV (SEQ ID NO:1), and VLPALP (SEQ ID NO:4). These are also capable of reducing production of NO by a cell. It is herein also provided to use a composition that comprises at least two oligopeptides or functional analogues thereof, each capable of down-regulation NFkappaB, and thereby reducing production of NO and/or TNF-alpha by a cell, in particular wherein the at least two oligopeptides are selected from the group LQGV (SEQ ID NO:1), AQGV (SEQ ID NO:2), and VLPALP (SEQ ID NO:4). Useful NFkappaB up-regulating peptides are VLPALPQ (SEQ ID NO:_), GVLPALP (SEQ ID NO:16), and MTRV (SEQ ID NO:20). As indicated, more gene-regulatory peptides may be found with an appropriate (bio)assay. A gene-regulatory peptide as used herein is preferably short. Preferably, such a peptide is three to fifteen amino acids long, and capable of modulating the expression of a gene, such as a cytokine, in a cell. In certain embodiments, a peptide is a signaling molecule that is capable of traversing the plasma membrane of a cell or, in other words, a peptide that is membrane-permeable, more preferably, wherein the lead peptide is three to nine amino acids long, most preferred wherein the lead peptide is four to six amino acids long.

Functional derivative or analogue herein relates to the signaling molecular effect or activity as, for example, can be measured by measuring nuclear translocation of a relevant transcription factor, such as NF-kappaB in an NF-kappaB assay, or AP-1 in an AP-1 assay, or by another method as provided herein. Fragments can be somewhat (i.e., one or two amino acids) smaller or larger on one or both sides, while still providing functional activity. Such a bioassay comprises an assay for obtaining information about the capacity or tendency of a peptide, or a modification thereof, to regulate expression of a gene. A scan with, for example, a 15-mer, or a 12-mer, or a 9-mer, or a 8-mer, or a 7-mer, or a 6-mer, or a 5-mer, or a 4-mer or a 3-mer peptide can yield valuable information on the linear stretch of amino acids that form an interaction site and allows identification of gene-regulatory peptides that have the capacity or tendency to regulate gene expression. Gene-regulatory peptides can be modified to modulate their capacity or tendency to regulate gene expression, which can be easily assayed in an in vitro bioassay such as a reporter assay. For example, some amino acid at some position can be replaced with another amino acid of similar or different properties. Alanine (Ala)-replacement scanning, involving a systematic replacement of each amino acid by an Ala residue, is a suitable approach to modify the amino acid composition of a gene-regulatory peptide when in a search for a signaling molecule capable of modulating gene expression. Of course, such replacement scanning or mapping can be undertaken with amino acids other than Ala as well, and also with D-amino acids. In one embodiment, a peptide derived from a naturally occurring polypeptide is identified as being capable of modulating gene expression of a gene in a cell. Subsequently, various synthetic Ala-mutants of this gene-regulatory peptide are produced. These Ala-mutants are screened for their enhanced or improved capacity to regulate expression of a gene compared to gene-regulatory polypeptide.

Furthermore, a gene-regulatory peptide, or a modification or analogue thereof, can be chemically synthesized using D- and/or L-stereoisomers. For example, a gene-regulatory peptide that is a retro-inverso of an oligopeptide of natural origin is produced. The concept of polypeptide retro-inversion (assemblage of a natural L-amino acid-containing parent sequence in reverse order using D-amino acids) has been applied successfully to synthetic peptides. Retro-inverso modification of peptide bonds has evolved into a widely used peptidomimetic approach for the design of novel bioactive molecules, which has been applied to many families of biologically active peptides. The sequence, amino acid composition and length of a peptide will influence whether correct assembly and purification are feasible. These factors also determine the solubility of the final product. The purity of a crude peptide typically decreases as the length increases. The yield of peptide for sequences less than 15 residues is usually satisfactory, and such peptides can typically be made without difficulty.

The overall amino acid composition of a peptide is an important design variable. A peptide's solubility is strongly influenced by composition. Peptides with a high content of hydrophobic residues, such as Leu, Val, Ile, Met, Phe and Trp, will either have limited solubility in aqueous solution or be completely insoluble. Under these conditions, it can be difficult to use the peptide in experiments, and it may be difficult to purify the peptide if necessary. To achieve a good solubility, it is advisable to keep the hydrophobic amino acid content below 50% and to make sure that there is at least one charged residue for every five amino acids. At physiological pH Asp, Glu, Lys, and Arg all have charged side chains. A single conservative replacement, such as replacing Ala with Gly, or adding a set of polar residues to the N- or C-terminus, may also improve solubility. Peptides containing multiple Cys, Met, or Trp residues can also be difficult to obtain in high purity partly because these residues are susceptible to oxidation and/or side reactions. If possible, one should choose sequences to minimize these residues. Alternatively, conservative replacements can be made for some residues. For instance, Norleucine can be used as a replacement for Met, and Ser is sometimes used as a less reactive replacement for Cys. If a number of sequential or overlapping peptides from a protein sequence are to be made, making a change in the starting point of each peptide may create a better balance between hydrophilic and hydrophobic residues. A change in the number of Cys, Met, and Trp residues contained in individual peptides may produce a similar effect.

In another embodiment of the invention, a gene-regulatory peptide capable of modulating gene expression is a chemically modified peptide. A peptide modification includes phosphorylation (e.g., on a Tyr, Ser or Thr residue), N-terminal acetylation, C-terminal amidation, C-terminal hydrazide, C-terminal methyl ester, fatty acid attachment, sulfonation (tyrosine), N-terminal dansylation, N-terminal succinylation, tripalmitoyl-S-Glyceryl Cysteine (PAM3 Cys-OH) as well as farnesylation of a Cys residue. Systematic chemical modification of a gene-regulatory peptide can, for example, be performed in the process of gene-regulatory peptide optimalization.

Synthetic peptides can be obtained using various procedures known in the art. These include solid phase peptide synthesis (SPPS) and solution phase organic synthesis (SPOS) technologies. SPPS is a quick and easy approach to synthesize peptides and small proteins. The C-terminal amino acid is typically attached to a cross-linked polystyrene resin via an acid labile bond with a linker molecule. This resin is insoluble in the solvents used for synthesis, making it relatively simple and fast to wash away excess reagents and by-products.

The peptides as mentioned in this document such as LQG, AQG, LQGV (SEQ ID NO:1), AQGV (SEQ ID NO:2), LQGA (SEQ ID NO:3), VLPALP (SEQ ID NO:4), ALPALP (SEQ ID NO:5), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLPALA (SEQ ID NO:12), VLPALPQ (SEQ ID NO:13), VLAALPQ (SEQ ID NO:14), VLPALPA (SEQ ID NO:15), GVLPALP (SEQ ID NO:16), VVCNYRDVRFESIRLPGCPRGVNPVVSYAVALSCQCAL (SEQ ID NO:_), RPRCRPINATLAVEKEGCPVCITVNTTICAGYCPT (SEQ ID NO:_), SKAPPPSLPSPSRLPGPS (SEQ ID NO:_), LQGVLPALPQVVC (SEQ ID NO:17), SIRLPGCPRGVNPVVS (SEQ ID NO:_), LPGCPRGVNPVVS (SEQ ID NO:18), LPGC (SEQ ID NO:19), MTRV (SEQ ID NO:20), MTR, and VVC were prepared by solid-phase synthesis using the fluorenylmethoxycarbonyl (Fmoc)/tert-butyl-based methodology with 2-chlorotrityl chloride resin as the solid support. The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP.

Upon completion of the synthesis, the peptide resin was treated with a mixture of trifluoroacetic acid (TFA)/H₂O/triisopropylsilane (TIS) 95:2.5:2.5. After 30 minutes, TIS was added until decolorization. The solution was evaporated in vacuo and the peptide precipitated with diethylether. The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/minute; absorbance was detected from 190-370 nm. There were different gradient systems used. For example, for peptides LQG and LQGV: ten minutes 100% A followed by linear gradient 0-10% B in 50 minutes. For example, for peptides VLPALP and VLPALPQ: five minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The elute was concentrated and lyophilized in 28 hours. Peptides later were prepared for use by dissolving them in PBS.

RAW 264.7 macrophages, obtained from American Type Culture Collection (Manassas, Va.), were cultured at 37° C. in 5% CO₂ using DMEM containing 10% FBS and antibiotics (100 U/ml of penicillin, and 100 μg/ml streptomycin). Cells (1×10⁶/ml) were incubated with peptide (10 μg/ml) in a volume of 2 ml. After eight hours of culture, cells were washed and prepared for nuclear extracts.

Nuclear extracts and EMSA were prepared according to Schreiber et al. methods (Schrieber et al. 1989, Nucleic Acids Research 17). Briefly, nuclear extracts from peptide stimulated or nonstimulated macrophages were prepared by cell lysis followed by nuclear lysis. Cells were then suspended in 400 μl of buffer (10 mM HEPES (pH 7.9), 10 mM KCl, 0.1 mM KCL, 0.1 mM EDTA, 0.1 mM EGTA, 1 mM DTT, 0.5 mM PMSF and protease inhibitors), vigorously vortexed for 15 seconds, left standing at 4° C. for 15 minutes, and centrifuged at 15,000 rpm for two minutes. The pelleted nuclei were resuspended in buffer (20 mM HEPES (pH 7.9), 10% glycerol, 400 mM NaCl, 1 mM EDTA, 1 mM EGTA, 1 mM DTT, 0.5 mM PMSF and protease inhibitors) for 30 minutes on ice, then the lysates were centrifuged at 15,000 rpm for two minutes. The supernatants containing the solubilized nuclear proteins were stored at −70° C. until used for the Electrophoretic Mobility Shift Assays (EMSA).

Electrophoretic mobility shift assays were performed by incubating nuclear extracts prepared from control (RAW 264.7) and peptide treated RAW 264.7 cells with a 32P-labeled double-stranded probe (5′ AGCTCAGAGGGGGACTTTCCGAGAG 3′ (SEQ ID NO:_) synthesized to represent the NF-kappaB binding sequence. Shortly, the probe was end-labeled with T4 polynucleotide kinase according to manufacturer's instructions (Promega, Madison, Wis.). The annealed probe was incubated with nuclear extract as follows: in EMSA, binding reaction mixtures (20 μl) contained 0.25 μg of poly(dI-dC) (Amersham Pharmacia Biotech) and 20,000 rpm of 32P-labeled DNA probe in binding buffer consisting of 5 mM EDTA, 20% Ficoll, 5 mM DTT, 300 mM KCl and 50 mM HEPES. The binding reaction was started by the addition of cell extracts (10 μg) and was continued for 30 minutes at room temperature. The DNA-protein complex was resolved from free oligonucleotide by electrophoresis in a 6% polyacrylamide gel. The gels were dried and exposed to x-ray films.

The transcription factor NF-kB participates in the transcriptional regulation of a variety of genes. Nuclear protein extracts were prepared from LPS and peptide treated RAW264.7 cells or from LPS treated RAW264.7 cells. In order to determine whether the peptide modulates the translocation of NF-kB into the nucleus, on these extracts EMSA was performed. Here we see that indeed some peptides are able to modulate the translocation of NF-kB since the amount of labeled oligonucleotide for NF-kB is reduced. In this experiment peptides that show the modulation of translocation of NF-kB are: VLPALPQVVC (SEQ ID NO:_), LQGVLPALPQ (SEQ ID NO:_, LQG, LQGV (SEQ ID NO:1), GVLPALPQ (SEQ ID NO:_, VLPALP (SEQ ID NO:4), VLPALPQ (SEQ ID NO:_), GVLPALP (SEQ ID NO:16), VVC, MTRV (SEQ ID NO:20), MTR.

RAW 264.7 mouse macrophages were cultured in DMEM, containing 10% or 2% FBS, penicillin, streptomycin and glutamine, at 37° C., 5% CO₂. Cells were seeded in a 12-wells plate (3×10⁶ cells/ml) in a total volume of 1 ml for two hours and then stimulated with LPS (E. coli 026:B6; Difco Laboratories, Detroit, Mich., USA) and/or peptide (1 microgr/ml). After 30 minutes of incubation, plates were centrifuged and cells were collected for nuclear extracts. Nuclear extracts and EMSA were prepared according to Schreiber et al. Cells were collected in a tube and centrifuged for five minutes at 2000 rpm (rounds per minute) at 4° C. (Universal 30 RF, Hettich Zentrifuges). The pellet was washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five microliters 10% NP-40 was added and the sample was centrifuged (two minutes, 4000 rpm, 4° C.). The supernatant (cytoplasmic fraction) was collected and stored at −70° C. The pellet, which contains the nuclei, was washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples were left to shake at 4° C. for at least 60 minutes. Finally the samples were centrifuged and the supernatant (nucleic fraction) was stored at −70° C.

Bradford reagent (Sigma) was used to determine the final protein concentration in the extracts. For Electrophoretic mobility shift assays an oligonucleotide representing NF-κB binding sequence (5′-AGC TCA GAG GGG GAC TTT CCG AGA G-3′ (SEQ ID NO:_)) was synthesized. Hundred pico mol sense and antisense oligo were annealed and labeled with γ-³²P-dATP using T4 polynucleotide kinase according to the manufacturer's instructions (Promega, Madison, Wis.). Nuclear extract (5-7.5 μg) was incubated for 30 minutes with 75,000 cpm probe in binding reaction mixture (20 microliters) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. The DNA-protein complex was resolved from free oligonucleotide by electrophoresis in a 4-6% polyacrylamide gel (150 V, two to four hours). The gel was then dried and exposed to x-ray film. The transcription factor NF-kB participates in the transcriptional regulation of a variety of genes. Nuclear protein extracts were prepared from either LPS (1 mg/ml), peptide (1 mg/ml) or LPS in combination with peptide treated and untreated RAW264.7 cells. In order to determine whether the peptides modulate the translocation of NF-kB into the nucleus, on these extracts EMSA was performed. Peptides are able to modulate the basal as well as LPS-induced levels of NF-kB. In this experiment peptides that show the inhibition of LPS-induced translocation of NF-kB are: VLPALPQVVC (SEQ ID NO:_), LQGVLPALPQ (SEQ ID NO:_), LQG, LQGV (SEQ ID NO:1), GVLPALPQ (SEQ ID NO:_), VLPALP (SEQ ID NO:4), VVC, MTR and circular LQGVLPALPQVVC (SEQ ID NO:_). Peptides that in this experiment promote LPS-induced translocation of NF-kB are: VLPALPQ (SEQ ID NO:13), GVLPALP (SEQ ID NO:16), and MTRV (SEQ ID NO:20). Basal levels of NF-kB in the nucleus were decreased by VLPALPQVVC (SEQ ID NO:_), LQGVLPALPQ (SEQ ID NO:_), LQG and LQGV (SEQ ID NO:1) while basal levels of NF-kB in the nucleus were increased by GVLPALPQ (SEQ ID NO:_), VLPALPQ (SEQ ID NO:_), GVLPALP (SEQ ID NO:_), VVC, MTRV (SEQ ID NO:20), MTR and LQGVLPALPQVVC (SEQ ID NO:_). In other experiments, QVVC (SEQ ID NO:_) also showed the modulation of translocation of NF-kB into nucleus (data not shown).

### Further Modes of Identification of Gene-Regulatory Peptides by NFkB Analysis

Cells: Cells will be cultured in appropriate culture medium at 37° C., 5% CO₂. Cells will be seeded in a 12-well plate (usually 1×10⁶ cells/ml) in a total volume of 1 ml for two hours and then stimulated with regulatory peptide in the presence or absence of additional stimuli such as LPS. After 30 minutes of incubation, plates will be centrifuged and cells are collected for cytosolic or nuclear extracts.

Nuclear Extracts: Nuclear extracts and EMSA could be prepared according to Schreiber et al. method (Schriber et al. 1989, Nucleic Acids Research 17). Cells are collected in a tube and centrifuged for five minutes at 2000 rpm (rounds per minute) at 4° C. (Universal 30 RF, Hettich Zentrifuges). The pellet is washed with ice-cold Tris buffered saline (TBS pH 7.4) and resuspended in 400 μl of a hypotonic buffer A (10 mM HEPES pH 7.9, 10 mM KCl, 0.1 mM EDTA, 0.1 mM EGTA, 1 mM DTT, 0.5 mM PMSF and protease inhibitor cocktail (Complete™ Mini, Roche) and left on ice for 15 minutes. Twenty-five microliters 10% NP-40 is added and the sample is centrifuged (two minutes, 4000 rpm, 4° C.). The supernatant (cytoplasmic fraction) was collected and stored at −70° C. for analysis. The pellet, which contains the nuclei, is washed with 50 μl buffer A and resuspended in 50 μl buffer C (20 mM HEPES pH 7.9, 400 mM NaCl, 1 mM EDTA, 1 mM EGTA, 1 mM DTT, 0.5 mM PMSF and protease inhibitor cocktail and 10% glycerol). The samples are left to shake at 4° C. for at least 60 minutes. Finally the samples are centrifuged and the supernatant (nucleic fraction) is stored at −70° C. for analysis.

Bradford reagent (Sigma) could be used to determine the final protein concentration in the extracts.

EMSA: For Electrophoretic mobility shift assays an oligonucleotide representing NF-κB binding sequence such as (5′-AGC TCA GAG GGG GAC TTT CCG AGA G-3′ (SEQ ID NO:_) are synthesized. Hundred pico mol sense and antisense oligo are annealed and labeled with γ-³²P-dATP using T4 polynucleotide kinase according to the manufacturer's instructions (Promega, Madison, Wis.). Cytosolic extract or nuclear extract (5-7.5 μg) from cells treated with regulatory peptide or from untreated cells is incubated for 30 minutes with 75,000 cpm probe in binding reaction mixture (20 μl) containing 0.5 μg poly dI-dC (Amersham Pharmacia Biotech) and binding buffer BSB (25 mM MgCl₂, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complex are resolved from free oligonucleotide by electrophoresis in a 4-6% polyacrylamide gel (150 V, two to four hours). The gel is then dried and exposed to x-ray film. Peptides can be biotinylated and incubated with cells. Cells are then washed with phosphate-buffered saline, harvested in the absence or presence of certain stimulus (LPS, PHA, TPA, anti-CD3, VEGF, TSST-1, VIP or know drugs, etc.). After culturing cells are lysed and cells lysates (whole lysate, cytosolic fraction or nuclear fraction) containing 200 micro gram of protein are incubated with 50 miroliters Neutr-Avidin-plus beads for one hour at 4° C. with constant shaking. Beads are washed five times with lysis buffer by centrifugation at 6000 rpm for one minute. Proteins are eluted by incubating the beads in 0.05 N NaoH for one minute at room temperature to hydrolyze the protein-peptide linkage and analyzed by SDS-polyacrylamide gel electrophoresis followed by immunoprecipitated with agarose-conjugated anti-NF-kB subunits antibody or immunoprecipitated with antibody against to be studied target.

After hydrolyzing the protein-peptide linkage, the sample could be analyzed on HPLS and mass-spectrometry. Purified NF-kB subunits or cell lysate interaction with biotinylated regulatory peptide can be analyzed on biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-kB transfected (reporter gene assay) cells and gene array technology can be used to determine the regulatory effects of peptides.

HPLC and mass-spectrometry analysis: Purified NF-kB subunit or cytosolic/nuclear extract is incubated in the absence or presence of (regulatory) peptide is diluted (2:1) with 8 N guanidinium chloride and 0.1% trifluoracetic acid, injected into a reverse-phase HPLC column (Vydac C18) equilibrated with solvent A (0.1% trifluoroacetic acid), and eluted with a gradient of 0 to 100% eluant B (90% acetonitrile in solvent A). Factions containing NF-kB subunit are pooled and concentrated. Fractions are then dissolved in appropriate volume and could be analyzed on mass-spectrometry.

In this study we demonstrate that LQGV (SEQ ID NO:1), AQGV (SEQ ID NO:2), and LAGV (SEQ ID NO:10), administrated after the induction of hemorrhagic shock in rats, significantly reduced TNF-α and IL-6 plasma levels, which is associated with reduced TNF-α and IL-6 mRNA transcript levels in the liver. This indicates that LQGV (SEQ ID NO:1), AQGV (SEQ ID NO:2), and LAGV (SEQ ID NO:10) may have therapeutic potential with beneficial effects on systemic inflammation, thereby reducing organ integrity/function, which is associated with shock and SIRS often seen with severe burns patients.

### Materials and Methods

Adult Male specific pathogen-free Wistar rats (Harlan CPB, Zeist, The Netherlands), weighing 350400 g were used after a minimum seven-day acclimation period. The animals were housed under barrier conditions and kept at 25° C. with a twelve-hour light/dark cycle. Rats were allowed free access to water and chow (−). All procedures were performed in accordance with the Principles of Laboratory Animal Care (NIH publication No. 86-23, revised 1985) under a protocol approved by the Committee on Animal Research of the Erasmus University (protocol EUR 365).

The rats were fasted overnight but were allowed free access to water before the experiment. Subsequent to endotracheal intubation the rats were mechanically ventilated with an isofluorane (−) N₂O/O₂ mixture at 60 breaths/minute. Body temperature was continuously maintained at 37.5° C. by placing the animals on a thermo controlled “half-pipe” (UNO, The Netherlands). Polyethylene tubes (PE-50, Becton Dickinson; St. Michielsgestel, The Netherlands) were flushed with heparin and placed via the right carotid artery in the aorta and in the right internal jugular vein. The animals received no heparin before or during the experiment.

Mean arterial pressures (MAP) was measured using transducers (Becton Dickinson) that were connected in line to an electronic recorder (Hewlett Packard, 78354-A Germany) for electronically calculated mean pressures and continuous measurement of the animal's blood pressure. Under semi-sterile conditions a median laparotomy was performed and ultrasonic perivascular flow probes (Transonic Systems Inc, Maastricht, The Netherlands) were placed on the common hepatic artery and the portal vein. A supra pubic catheter was placed to monitor the urine production during and after resuscitation.

After an acclimatization period of 20 minutes, the rats were randomized into the following five groups:

Hemorrhagic shock group were bled within ten minutes to a mean arterial pressure (MAP) of 40 mmHg and maintained at this level for 60 minutes by withdrawing or re-infusing shed blood as needed. Thereafter, the animals were resuscitated with plus minus four times the volume of the withdrawn blood over 30 minutes with a 0.9% NaCl solution.

The hemorrhagic shock group+peptide A (LAGV (SEQ ID NO:10); one-letter amino acid code) underwent the same procedure as the hemorrhagic shock group but received a single bolus injection of 5 mg/kg peptide A intravenously 30 minutes after the induction of shock.

The hemorrhagic shock group+peptide B (AQGV (SEQ ID NO:2)) underwent the same procedure as the hemorrhagic shock group and received a single bolus injection of 5 mg/kg peptide B intravenously, 30 minutes after the induction of shock.

The hemorrhagic shock group+peptide C (LQGV (SEQ ID NO:1)) underwent the same procedure as the hemorrhagic shock group and received a single bolus injection of 5 mg/kg peptide C intravenously, 30 minutes after the induction of shock.

Sham group underwent the same procedure as the hemorrhagic shock group without performing the hemorrhage or administration of any kind of peptides.

The hepatic arterial blood flow (QHA) and hepatic portal venous blood flow (QVP) were measured with transit time ultrasonic perivascular flow probes, connected to an ultrasonic meter (T201; Transonic Systems, Inc., Maastricht, NL). Systemic and hepatic hemodynamics were continuously measured. At regular time points arterial blood samples were taken. The animals were euthanized by withdrawal of arterial blood via the carotid artery.

### Blood, Tissue, and Cell Harvesting Procedure

Plasma collection and storage: Whole arterial blood was obtained at −15, 30, 60, 90, 120, 150 and 180 minutes after induction of shock via the right carotid artery and collected in duplo. 0.2 ml was placed in tubes (Eppendorf EDTA KE/1.3) to be assayed in the coulter counter (−). 0.5 ml was placed in Minicollect tubes (Bio-one, Greiner) centrifuged for five minutes, immediately frozen, and stored at −80° C., until assayed. All assays were corrected for the hematocrit.

Measurement of cytokines (still in progress): The levels of IL-6, and IL-10 in the serum were determined by an ELISA (R&D Systems Europe Ltd) according to the manufacturer's instructions.

Histology (still in progress): The alterations in lung, liver, sigmoid and small bowel morphology were examined in sham-operated animals, in animals after trauma-hemorrhage and in animals after trauma-hemorrhage treated with peptide A, B or C. All tissues were collected in duplo. One part was harvested and fixed in formalin (Sigma) and later embedded in paraffin. The other part was placed in tubes (NUNC Cryo Tube™ Vials), quick frozen in liquid nitrogen and stored at −80° C. until assayed.

### Results

Mean Arterial Pressure: MAP dropped in all shock groups significant during the shock phase compared to the control group.

Hematocrit: The hematocrit following trauma-hemorrhage was similar in the different peptide A, B and C treated and non-treated groups. During the shock phase there was a difference of hematocrit in the control group in comparison with the other groups. From the resuscitation phase (90 minutes) there was no significant difference in hematocrit among the control, trauma-hemorrhage, and peptide groups.

Leukocyte Recruitment: During trauma-hemorrhage the leukocytes dropped from 100% at T0 in all groups to a minimum of 40.0±11.9%, 42.0±8.7%, 47.3±12.4%, 38.2±7.4% in respectively the non-treated, peptide A treated, peptide B treated and peptide C treated groups because of leukocyte accumulation in the splanchnic microcirculation. There was a significant difference in leukocyte concentration between all treated and non-treated trauma-hemorrhage groups, and the control group during the shock phase. No significant difference was noticed between the peptide A, B or C treated animals and the non-treated animals.

Blood Concentrations of Macrophages and Granulocytes: At 180 minutes after the onset of trauma-hemorrhage, concentrations of circulating macrophages (M_{Φ}) and granulocytes were significant lower in the peptide B and C treated animals compared with the corresponding experimental group. Blood levels of circulating M_{Φ} and granulocytes were 5,556±1,698 10⁹/1 in sham-operated animals whereas blood levels were 6,329±1,965 10⁹/1 after trauma-hemorrhage, and decreased by 29.9% after administration of peptide B (4,432±0.736 10⁹/1) and 39.2% after administration of peptide C (3,846±0.636 10⁹/1) compared with concentrations after trauma-hemorrhage.

Arterial Hepatic Blood Flow: There was a decrease in the arterial hepatic blood flow in the shock group (18.3±14.3%) and in the peptide A (21.3±9.1%), B (18.1±9.0%) and C (21.2±8.6%) group during the shock period compared with the control group (102.6±23.5%). An increase in blood flow was observed during the reperfusion in the hepatic artery of the shock group (128.9±75.4%) compared with control animals (83.7±24.2%) and the animals treated with peptide B (78.4±28.3%).

Trauma-hemorrhage results in hypoxic stress owing to the absolute reduction in circulating blood volume. In contrast, sepsis is an inflammatory state mainly mediated by bacterial products. It is interesting that these divergent insults reveal similar pathophysiologic alterations in terms of the splanchnic circulation.

Hemorrhagic shock significantly increases leukocyte accumulation in the splanchnic microcirculation owing to the up-regulation of P selectin. The expression of intercellular adhesion molecule within the intestinal muscular vasculature after hemorrhagic shock promotes the local recruitment of leukocytes, and this inflammatory response is accompanied by subsequent impairment of intestinal function.

The adhesion and extravasation of neutrophils not only contribute to the inflammatory response in the splanchnic tissue bed but also induce intestinal microcirculatory failure and dysfunction after severe stress. This is mediated by the induced expression of adhesion molecules, such as selectins and endothelial cell adhesion molecules, on the surface of neutrophils and endothelial cells.

In our shock experiments, leukocyte concentration significant decreases during hemorrhagic shock compared to the control animals. However a single dose of peptide B or C administered during resuscitation, decreased concentrations of circulating macrophages and granolocytes 120 minutes after the onset of hemorrhagic shock compared to the non-treated animals.

Because some female sex hormones effectively protect the organs from circulatory failure after various adverse circulatory conditions, numerous studies have been performed to clarify the molecular mechanism of, for example, estradiol action with regard to tissue circulation. In this study, a single dose of peptide was administered following trauma-hemorrhage and various parameters were measured at three hours following the induction of sepsis. Treatment with peptides improved or restored immune functional parameters and cardiovascular functions. Therefore, our results show that administration of short oligopeptides (NMPFs) is beneficial in the treatment of critically ill trauma victims experiencing hemorrhagic shock.

### EXAMPLE 2

BACKGROUND: Hemorrhagic shock followed by resuscitation induces a massive pro-inflammatory response, which may culminate into severe inflammatory response syndrome, multiple organ failure and finally death. Treatments aimed at inhibiting the effects of pro-inflammatory cytokines are only effective when initiated before the onset of hemorrhagic shock, which severely limits their clinical application.

AIM: We investigated whether the administration of synthetic hCG-related oligopeptides (LQGV (SEQ ID NO:1), AQGV (SEQ ID NO:2), and LAGV (SEQ ID NO:10)) 30 minutes after induction of hemorrhagic shock reduced the inflammatory response.

METHODS: Rats were bled to 50% of baseline mean arterial pressure and one hour later resuscitated by autologous blood transfusion. Thirty minutes after onset of hemorrhagic shock, experimental groups received either one of the synthetic hCG-related oligopeptides (LQGV (SEQ ID NO:1), AQGV (SEQ ID NO:2), and LAGV (SEQ ID NO:10)) or 0.9% NaCl solution. TNF-α and IL-6 plasma levels were determined at fixed time points before and after onset of hemorrhagic shock. Liver, lungs, ileum and sigmoid mRNA levels for TNF-α, IL-6 and ICAM-1 were determined 180 minutes after onset of hemorrhage.

RESULTS: Treatment with either one of the three hCG-related oligopeptides (LQGV (SEQ ID NO:1), AQGV (SEQ ID NO:2), and LAGV (SEQ ID NO:10)) efficiently reduced TNF-α and IL-6 plasma levels as well as TNF-α and IL-6 mRNA transcript levels in the liver.

CONCLUSION: Considering these powerful effects of hCG-related oligopeptides during severe hemorrhagic shock, they may have therapeutic potential with beneficial effects on the hyper inflammation, thereby reducing the late life threatening tissue- and organ-damage that is associated with severe hemorrhagic shock, which can be a consequence of surgery.

INTRODUCTION: In hemorrhagic shock there is massive blood loss, which cannot be compensated by the body without treatment. The primary treatment of hemorrhagic shock is to control bleeding and restore intravascular volume to improve tissue perfusion. This treatment induces an inflammatory response, which may culminate into a severe inflammatory response and finally multiple organ dysfunction syndrome (MODS).^{[1, 2, 3]} In addition, approximately 40% of patients develop sepsis as a result of trauma-hemorrhage.^{[3]} Sepsis and MODS are the leading causes of death in critically ill patients on the intensive care unit all over the world with mortality rates of about 50%.^{[4, 5]}

The severe inflammatory response due to trauma-hemorrhage is characterized by increased expression of adhesion molecules, such as intracellular adhesion molecule-1 (ICAM-1) and vascular cell adhesion molecule-1 (VCAM-1), on sinusoidal endothelial cells and hepatocytes. Furthermore, increased levels of pro-inflammatory cytokines are found systemically and locally in liver, lungs and intestine.^{[6, 7, 8, 9]} The pro-inflammatory cytokines produced are in particular tumor necrosis factor alpha (TNF-α), interleukin (IL)-1β and IL-6.^{[10, 11, 12]} These cytokines affect organ integrity/function directly, but also indirectly through secondary mediators, such as nitric oxide, thromboxanes, leukotrienes, platelet-activating factor, prostaglandins, and complement.^{[13, 14]} TNF-α also causes the release of tissue-factor by endothelial cells leading to fibrin deposition and disseminated intravascular coagulation.^{[15, 16]} Cells within the liver, mainly Kupffer cells, but also hepatocytes and sinusoidal endothelial cells, are considered as the main producers of these pro-inflammatory cytokines during hemorrhagic shock.^{[17]}

The last decade, researchers have focused on the modulation of the systemic inflammatory responses with therapeutic agents aiming at neutralizing the activity of cytokines, especially TNF-α.^{[18]} Other researchers used therapeutic agents aiming at the inhibition of TNF-α production.^{[19]} However, most of these therapeutic agents must be administered before the onset of hemorrhagic shock to achieve a therapeutic effect.^{[19]} Clearly, this is almost impossible in a clinical trauma-hemorrhage setting. Therefore, therapies initiated after the onset of severe trauma-hemorrhage and aiming at reducing the production of pro-inflammatory cytokine are more relevant to prevent the events leading to MODS.

During pregnancy, the maternal immune system tolerates the fetus by reducing the cell-mediated immune response while retaining normal humoral immunity.^{[20]} Also, clinical symptoms of cell-mediated autoimmune diseases regress in many patients during pregnancy.^{[20]} The hormone human chorionic gonadotropin (hCG) is mainly secreted by placental syncytiocytotrophoblasts during pregnancy and has been shown to be immunoregulatory.^{[21, 22, 23]} The β-subunit of hCG is degraded by specific proteolytic enzymes.^{[24]} This can lead to the release of several oligopeptides consisting of four to seven amino acids that, because of their role in regulation of physiological processes, are considered regulatory.^{[25]} We successfully demonstrated that synthetic hCG-related oligopeptides can inhibit the acute inflammatory response, disease severity, and mortality in high-dose lipopolysaccharide-induced systemic inflammatory response syndrome.^{[26]} Considering these powerful regulating effects of synthetic hCG-related oligopeptides on inflammation, we hypothesized that the administration of such regulatory oligopeptides after severe trauma-hemorrhage could inhibit the massive inflammatory response, associated with this condition. To this end, we used LQGV, which is part of the primary structure of loop two of the β-subunit of hCG, and two alanine replacement variants, namely AQGV and LAGV.

In this study we demonstrate that LQGV (SEQ ID NO:1), AQGV (SEQ ID NO:2), and LAGV (SEQ ID NO:10), administrated after the induction of hemorrhagic shock in rats, significantly reduced TNF-α and IL-6 plasma levels, which is associated with reduced TNF-α and IL-6 mRNA transcript levels in the liver. This indicates that LQGV (SEQ ID NO:1), AQGV (SEQ ID NO:2), and LAGV (SEQ ID NO:10) may have therapeutic potential with beneficial effects on systemic inflammation, thereby reducing organ integrity/function, which is associated with severe hemorrhagic shock.

### Materials and Methods

### Animals

Adult male specific pathogen-free Wistar rats (Harlan CPB, Zeist, The Netherlands), weighing 350-400 g were used. Animals were housed under barrier conditions at 25° C. with a twelve-hour light/dark cycle, and were allowed food and water ad libitum. The experimental protocol was approved by the Animal Experiment Committee under the Dutch Experiments on Animals Act and adhered to the rules laid down in this national law that serves the implementation of “Guidelines on the protection of experimental animals” by the Council of Europe (1986), Directive 86/609/EC.

hCG-related synthetic oligopeptides: The hCG-related oligopeptides (LQGV (SEQ ID NO:1), AQGV (SEQ ID NO:2), and LAGV (SEQ ID NO:10)) were synthesized by Ansynth Service B.V. (Roosendaal, The Netherlands) and dissolved in 0.9% NaCl at a concentration of 10 mg/ml.

Surgical procedures: Rats were food deprived overnight before the experiment, but were allowed water ad libitum. Rats were anesthetized using a mixture of N₂O/O₂ isoflurane (Pharmachemie B.V., Haarlem, the Netherlands). Body temperature was continuously maintained at 37.5° C. by placing the rats on a thermo controlled “half-pipe” (UNO, Rotterdam, The Netherlands). Endotracheal intubation was performed, and rats were ventilated at 60 breaths per minute with a mixture of N₂O/O₂ 2% isoflurane. Polyethylene tubes (PE-50, Becton Dickinson; St. Michielsgestel, The Netherlands) were flushed with heparin and placed via the right carotid artery in the aorta and in the right internal jugular vein. The rats received no heparin before or during the experiment.

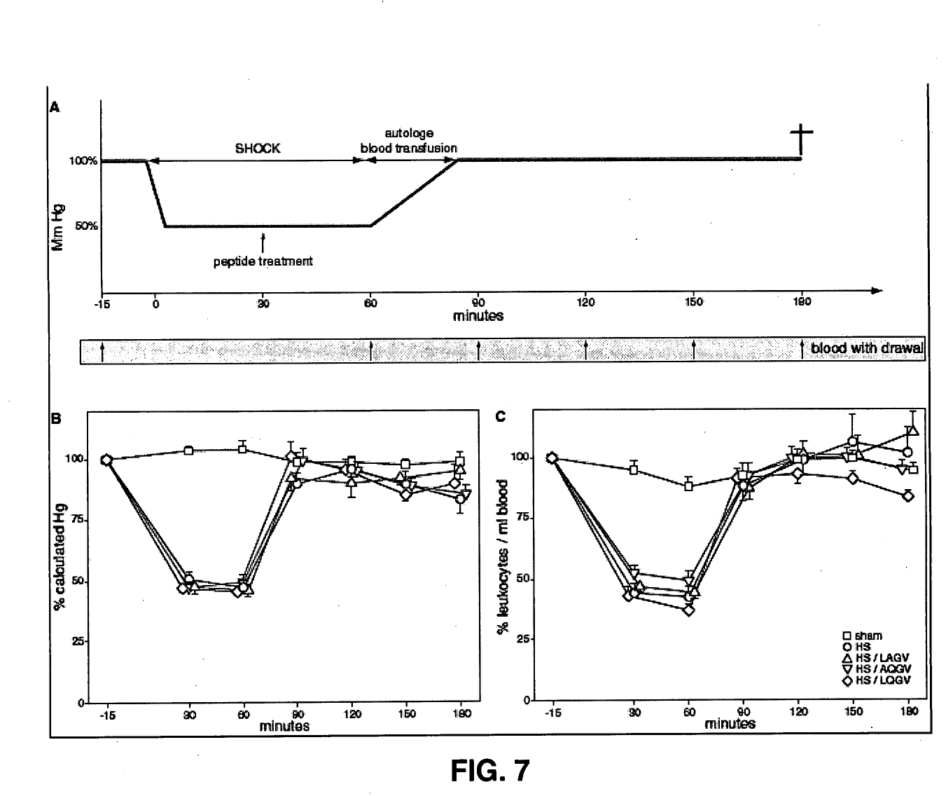

Experimental procedures: After an acclimatization period of 15 minutes, the rats were randomized into five different groups: 1) sham, 2) hemorrhagic shock (HS), 3) hemorrhagic shock with LQGV (SEQ ID NO:1) treatment (HS/LQGV (SEQ ID NO:1)), 4) hemorrhagic shock with AQGV (SEQ ID NO:2) treatment (HS/AQGV (SEQ ID NO:2)) and 5) hemorrhagic shock with LAGV treatment (HS/LAGV (SEQ ID NO:10)). Hemorrhagic shock was induced by blood withdrawal, reducing the circulating blood volume until a mean arterial pressure (MAP) of 50% of normal mmHg was reached. This level of hypotension was maintained for 60 minutes. After 30 minutes, rats received either a single bolus injection of 10 mg/kg LQGV (SEQ ID NO:1), AQGV (SEQ ID NO:2), LAGV (SEQ ID NO:10) or 0.9% NaCl solution. The peptides and dosage were based on previous studies, in which we performed dose-escalation experiments (manuscript in preparation). Sixty minutes after induction of hemorrhagic shock, rats were resuscitated by autologous blood transfusion over a period of 30 minutes and monitored for another 120 minutes after which they were sacrificed (FIG. 7A). Sham animals underwent the same surgical procedure as the hemorrhagic shock animals, but without performing hemorrhage and administration of peptides.

Plasma collection and storage: Arterial blood was obtained 15 minutes before and 30, 60, 90, 120, 150 and 180 minutes after onset of hemorrhage (FIG. 7A). After blood withdrawal, leukocyte numbers were determined using a coulter counter (Beckman Coulter, Mijdrecht, the Netherlands) and corrected for the hematocryte. Approximately, 0.3 ml of blood was placed into mini collect tubes (Greiner, Bio-one, Alphen a/d Rijn, the Netherlands), plasma was obtained by centrifugation (1500 r.p.m.; five minutes), immediately frozen, and stored at −80° C., until assayed.

Measurements of Mean arterial pressure: During the experiments, mean arterial pressure (MAP) was continuously measured using transducers (Becton Dickinson) that were connected in line to an electronic recorder (Hewlett Packard, 78354-A, Germany).

Tissue collection and storage: Liver, lungs, ileum and sigmoid were surgically removed at the end of the experiment, snap-frozen, and stored at −80° C., until assayed.

Measurement of cytokines: TNF-α and IL-6 plasma levels were determined by ELISA (R&D Systems Europe Ltd, Abingdon, UK), according to the manufacturer's instructions.

Evaluation of mRNA levels by real-time quantitative (RQ)-PCR: RNA was isolated using a QIAGEN kit (QIAGEN, Hilden, Germany), according to the manufacturer's instructions. TNF-α, IL-6 and ICAM-1 transcripts were determined by RQ-PCR using an Applied Biosystems 7700 PCR machine (Foster City, Calif., USA) as described previously.^{[27]} TNF-α, IL-6 and ICAM-1 expression was quantified by normalization against GAPDH. Primer probe combinations used are listed in Table 1.

Statistical analysis: Statistical analysis was performed using SPSS version 11 software (SPSS Inc., Chicago, Ill.). Inter group differences were analyzed with Kruskal-Wallis statistical test. If Kruskal-Wallis statistical testing resulted in a p<0.05, a Dunn's Multiple Comparison test was performed and p<0.05 was considered statistically significant.

### Results

Induction of hemorrhagic shock: Lowering the MAP to 50% of normal induced hemorrhagic shock, which was successfully maintained for 60 minutes in all four experimental groups (FIG. 7B). No change in MAP was observed in sham treated rats (FIG. 7B). A decrease in the percentage of blood leukocytes was observed in all four experimental groups after blood withdrawal (FIG. 7C). Sixty minutes after hemorrhagic shock, rats were resuscitated with there own blood to induce organ reperfusion, which was associated with a normalization of leukocyte level (FIG. 7C).

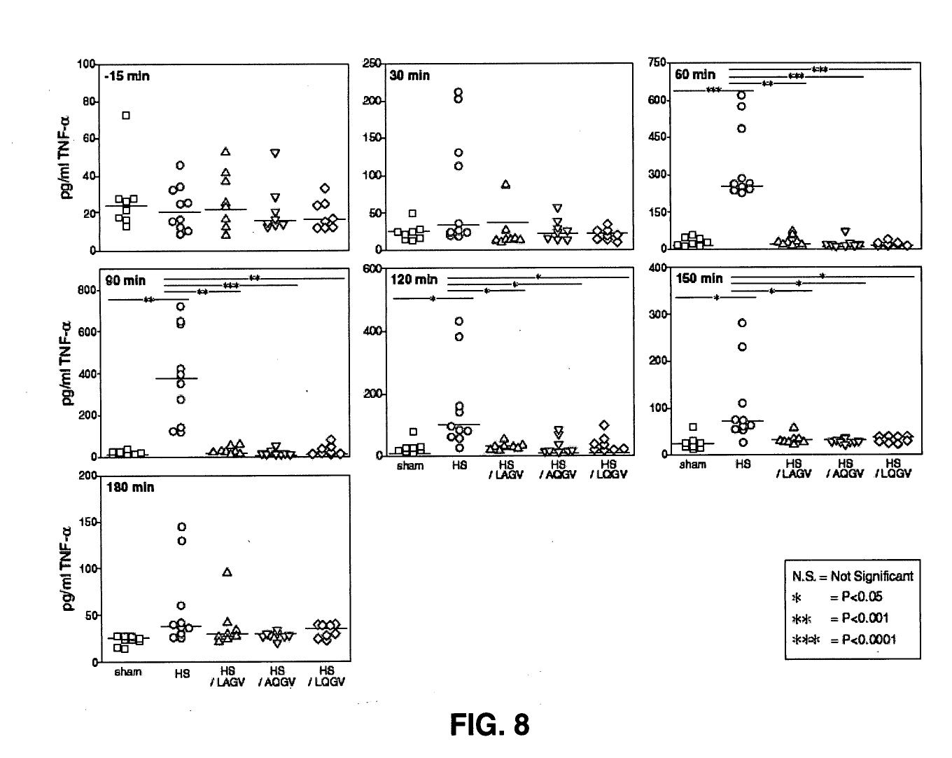

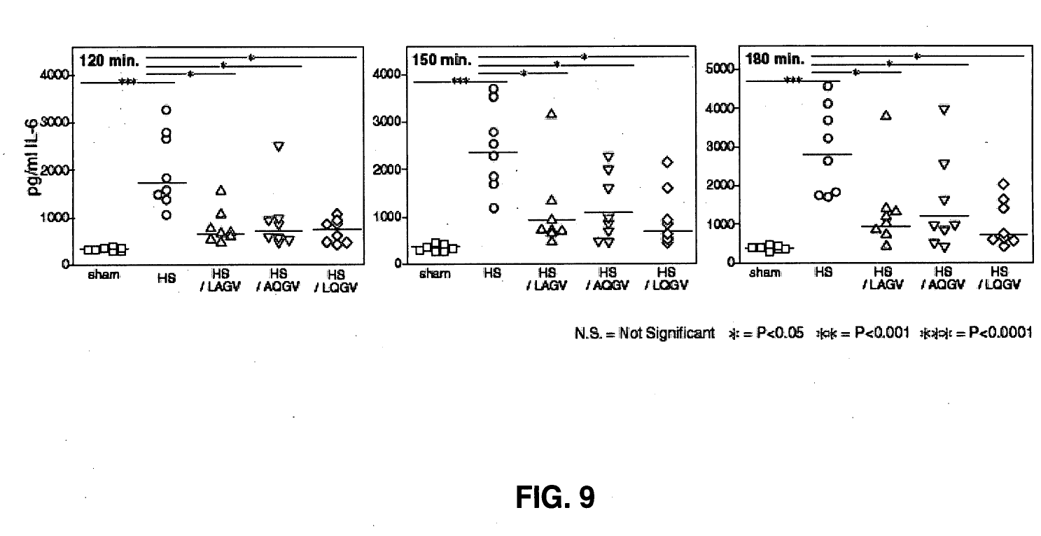

Oligopeptide treatment reduces pro-inflammatory cytokine plasma levels: The therapeutic capacity of three synthetic oligopeptides (LQGV (SEQ ID NO:1), AQGV (SEQ ID NO:2), LAGV (SEQ ID NO:10)) related to the primary structure of loop two of the β-subunit of hCG was evaluated in a rat hemorrhagic shock model. Before induction of hemorrhage, TNF-α plasma levels were comparable in all five groups (˜15-24 pg/ml) (FIG. 8). In the HS group, TNF-α levels started to increase thirty minutes after induction of hemorrhagic shock and were significantly increased after sixty minutes, as compared to the sham group (264 pg/ml vs 24 pg/ml, respectively; p<0.01). TNF-α levels reached a maximum of 374 pg/ml after 90 minutes in the HS group, after which levels declined again but always remaining increased compared to the sham group (FIG. 8). In contrast, none of the oligopeptide-treated HS groups (HS/LQGV (SEQ ID NO:1), HS/AQGV (SEQ ID NO:2), HS/LAGV (SEQ ID NO:10)) showed an increase in plasma TNF-α levels during the experiment (FIG. 8). IL-6 levels are known to increase at a later time-point than TNF-α after severe hemorrhagic shock.^{[11, 12]} Therefore, we determined IL-6 levels in blood samples collected 120, 150 and 180 minutes after the onset of hemorrhagic shock. In the HS group, IL-6 plasma levels were significantly increased as compared to sham group at 120 minutes (1704 pg/ml vs 338 pg/ml, respectively; p<0.001), at 150 minutes (2406 pg/ml vs 316 pg/ml, respectively; p<0.001) and at 180 minutes (2932 pg/ml vs 369 pg/ml, respectively; p<0.001) (FIG. 9). Although IL-6 levels tended to increase a little in the HS/oligopeptide treated rats as compared to sham treated rats, this never reached significance. Treatment with oligopeptides after hemorrhagic shock (HS/LQGV (SEQ ID NO:1), HS/AQGV (SEQ ID NO:2), HS/LAGV (SEQ ID NO:10)) resulted in a significant reduction of IL-6 plasma levels as compared to the non-treated hemorrhagic shock group (HS) (FIG. 9). These data demonstrate that treatment with a single dose of either LQGV (SEQ ID NO:1), AQGV (SEQ ID NO:2), or LAGV (SEQ ID NO:10) after induction of hemorrhagic shock results in a significant reduction of TNF-α and IL-6 plasma levels.

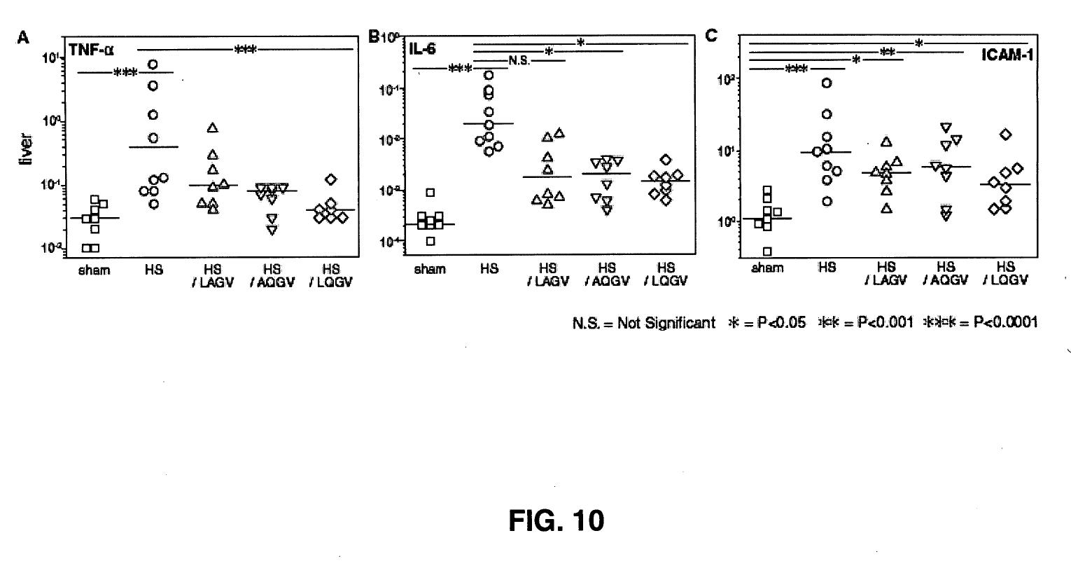

Oligopeptide treatment reduces TNF-α and IL-6 but not ICAM-1 mRNA levels in the liver: Because oligopeptide treatment clearly decreased the TNF-α and IL-6 plasma levels, we analyzed mRNA levels in liver, lungs, ileum and sigmoid tissues at 180 minutes after the onset of hemorrhagic shock. In the liver, TNF-α transcripts were significantly increased in the HS group as compared to the sham group. Oligopeptide treatment was associated with decreased TNF-α transcripts in the liver as compared to non-treated HS rats with only HS/LQGV (SEQ ID NO:1) showing a significant reduction as compared to HS (p<0.01; FIG. 10A).

In the HS group, IL-6 transcripts in the liver were increased ˜83 times as compared to the sham group (p<0.001; FIG. 10B). None of the oligopeptide treated groups showed an increase in IL-6 mRNA as compared to the sham treated group. LQGV (SEQ ID NO:1) and AQGV (SEQ ID NO:2) treatment resulted in a significant reduction in IL-6 mRNA transcripts as compared to the HS group (p<0.05; FIG. 10B).

ICAM-1 transcript levels in the liver were significantly increased in the HS group as compared to the sham group (FIG. 10C). Oligopeptide treatment during hemorrhagic shock (HS/LQGV (SEQ ID NO: 1), HS/AQGV (SEQ ID NO:2), HS/LAGV (SEQ ID NO:10)) did not affect the ICAM-1 transcript levels in the liver (FIG. 10C). In lungs, ileum and sigmoid tissue no significant differences could be detected between the various groups for TNF-α, IL-6 and ICAM-1 (data not shown). These data indicate that oligopeptide treatment following hemorrhagic shock decreases pro-inflammatory cytokine transcript levels in the liver but does not reduce ICAM-1 transcript levels.

### Discussion

In this study we used a rat model of hemorrhagic shock and demonstrated that administration of synthetic hCG-related oligopeptides (LQGV (SEQ ID NO:1), AQGV (SEQ ID NO:2), LAGV (SEQ ID NO:10)) 30 minutes after shock induction, efficiently reduces the pro-inflammatory cytokine levels associated with this condition. Our data demonstrate this to be the consequence of reduced expression of pro-inflammatory cytokine mRNA transcript levels in the liver.

Hemorrhagic shock is associated with an early adherence of leukocytes to the vascular endothelium as a result of a decreased blood volume.^{[28]} In our model a decrease in the percentage of leukocytes was detected in all four experimental groups after blood withdrawal. This indicates that all experimental groups experienced hemorrhagic-induced shock. Resuscitation resulted in an increase of the percentages of leukocytes in the experimental groups.

Hemorrhagic shock followed by resuscitation induces a severe inflammatory response, which is characterized by an exaggerated production of early pro-inflammatory cytokines, such as TNF-α, IL1β, and subsequently IL-6.^{[10, 11, 12]} TNF-α is a key mediator of the innate immune system that is crucial for the generation of a local protective immune response against infectious or non-infectious agents.^{[9]} However, uncontrolled massive TNF-α production is lethal, as it spreads via the bloodstream into other organs thereby inducing tissue damage and promoting the production of secondary pro-inflammatory mediators, such as IL6.^{[10, 11]}

Despite improvement in treatment strategies, trauma-hemorrhage patients may still develop severe inflammatory response that leads too MODS and finally death. Experimental treatment strategies aimed at neutralizing bioactive cytokines, such as monoclonal antibodies against TNF-α, have been successfully applied in several inflammatory disorders, including Crohn's disease and Rheumatoid Arthritis.^{[29, 30]} However, clinical studies using monoclonal antibodies against TNF-α showed no clinical effect in trauma-patients.^{[31]} It has been suggested that TNF-α neutralizing antibodies causes the accumulation of a large pool of TNF-α/anti-TNF-α pool, which act as a slow release reservoir that may lead to increased constant active TNF-α.^{[32]} Therefore, aiming at therapies that decrease the production of TNF-α and IL-6 may be more beneficial in limiting tissue damage and mortality rates in trauma-hemorrhage patients than neutralization of already produced cytokines.

In hemorrhagic shock, TNF-α is secreted within minutes after cellular stimulation, while production stops after three hours, and TNF-α plasma levels become almost undetectable.^{[9]} We demonstrate that hCG-related regulatory oligopeptides (LQGV, AQGV, LAGV), administered 30 minutes after the induction of hemorrhagic shock, significantly reduced TNF-α and IL-6 plasma levels. Whether the effect on IL-6 production is direct, or indirect due to reduced TNF-α plasma levels cannot be concluded from our data. Nevertheless, establishing a reduction of IL-6 is of clinical importance, because high IL-6 plasma levels correlate with poor outcome and decreased survival in patients with severe trauma and infection.^{[33, 34]} Cells within the liver, are considered as the main producers of pro-inflammatory cytokines during hemorrhagic shock.^{[17]} TNF-α and IL-6 transcript levels were significantly increased in the livers of the HS group. LQGV (SEQ ID NO:1), AQGV (SEQ ID NO:2), or LAGV (SEQ ID NO:10) treatment was associated with a reduction in TNF-α and IL-6 liver transcripts, which may be indicative of decreased transcriptional activation.

Another important characteristic of endothelial cells and hepatocytes during hemorrhagic shock is increased expression of the adhesion molecule ICAM-1.^{[7, 8]} Our study confirms the increased ICAM-1 expression in the liver after hemorrhagic shock. However, LQGV (SEQ ID NO:1), AQGV (SEQ ID NO:2), or LAGV (SEQ ID NO:10) treatment did not result in reduced ICAM-1 expression. This could be due to the inability of hCG-related oligopeptides to interfere with induction of ICAM-1 transcription. In lungs, ileum and sigmoid, we detected no effect of hemorrhagic shock on the induction of TNF-α, IL-6 and ICAM-1 transcripts. This confirms that the liver is the first organ in which the inflammatory response is initiated after hemorrhagic shock and fluid resuscitation.^{[15, 31, 32]}

In summary, a single administration of a synthetic hCG-related oligopeptide (LQGV (SEQ ID NO:1), AQGV (SEQ ID NO:2), LAGV (SEQ ID NO:10)) after the induction of severe trauma-hemorrhage reduces the subsequent pro-inflammatory response. These data suggest that these oligopeptides have therapeutic potential, in minimizing or preventing the late life threatening tissue- and organ-damage that is associated with SIRS seen with severe perioperative trauma.

### EXAMPLE 2

### Treatment of Severe Skin Inflammations Such as Seen Treatment with the Drug Imiquimod (ALDARA™)

To assess the activity of the various peptides with skin inflammations and tissue destruction seen, for example, with patients treated with the drug imiquimod (ALDARA™) an animal model was developed in which these inflammations are generated via topical application of the inflammatory agent to the skin of experimental mice. For this purpose mice were treated with 4% or 5% imiquimod. Imiquimod is an immune response modifier used in the treatment of skin cancers. It is manufactured as a 4% or 5% cream (ALDARA™). Imiquimod works by stimulating the immune system to release a number of chemicals called cytokines whereby it results in inflammation. The imiquimod is taken up by the so-called “toll-like receptor 7” on certain immune cells that are found in the outside part of the skin, the epidermis. Skin areas treated with imiquimod will become inflamed. The effects include itching, burning, redness, ulceration (sores), scabbing, flaking and pain, typically an example wherein medical treatment (an iatrogenic event) comprises destruction or lysis of a cell or tissue of the subject. Particularly the mice treated with the 5% cream developed the intense inflammatory skin lesions sometimes seen with iatrogenic wounds in skin cancer patients.

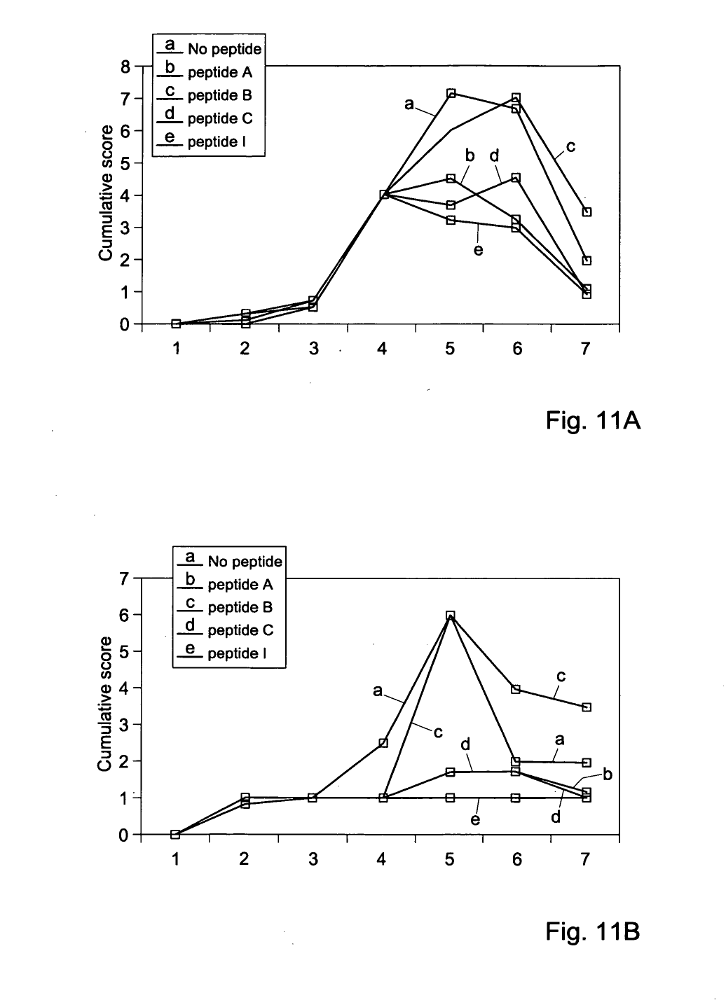

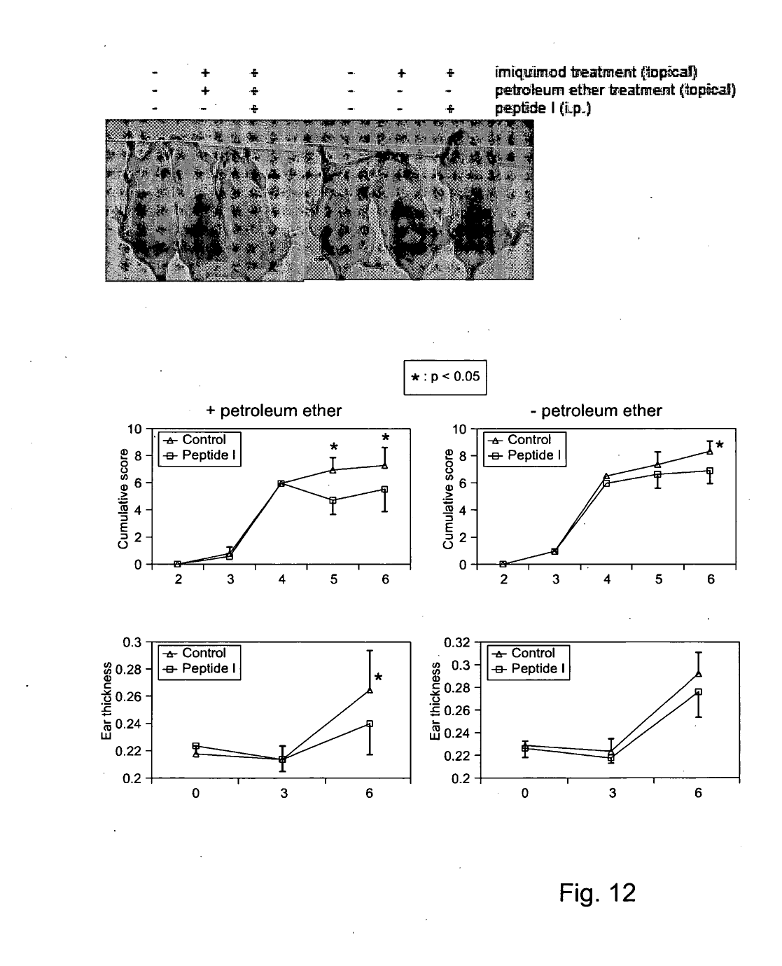

Peptides tested in this study were Peptide A (LAGV (SEQ ID NO:10)); Peptide B (AQGV (SEQ ID NO:2)); Peptide G (VLPALPQ (SEQ ID NO:13)) and Peptide I (LQGV (SEQ ID NO:1)). Peptides were given parenterally by intraperitoneal injection (i.p.). All peptides had beneficial activity on the imiquimod-induced skin lesions, especially after the lesions had occurred (see FIGS. 11 and 12). Treatment with petroleum ether to remove fat and scales of the imiquimod-induced lesions in one experiment improved the activity of peptide I (LQGV (SEQ ID NO:1)).

### REFERENCES

1. Vincent J. L., J. Carlet, and S. M. Opal, eds., The sepsis text, 2002.
2. Cohen J. The immunopathogenesis of sepsis. Nature 2002, 420:885-891.
3. Osborn T. M., J. K. Tracy, J. R. Dunne, M. Pasquale, and L. M. Napolitano. Epidemiology of sepsis in patients with traumatic injury. Crit. Care Med. 2004, 32(11):2234-40.
4. DS, R. Lefering, and C. E. Wade. Impact of hemorrhage on trauma outcome: an overview of epidemiology, clinical presentations, and therapeutic considerations. J. Trauma, 2006, 60(6):S3-11.
5. Muylle L. The role of cytokines in blood transfusion reactions. Blood Rev. 1995, 9:77-83.
6. Matsutani T., S. C. Kang, M. Miyashita, K. Sasajima, M. A. Choudhry, K. I. Bland, et al. Liver cytokine production and ICAM-1 expression following bone fracture, tissue trauma, and hemorrhage in middle-aged mice. Am. J. Physiol. Gastrointest. Liver Physiol. 2007, 292:268-74.
7. Rothoerl R. D., K. M. Schebesch, M. Kubitza, C. Woertgen, A. Brawanski, and A. L. Pina. ICAM-1 and VCAM-1 expression following aneurysmal subarachnoid hemorrhage and their possible role in the pathophysiology of subsequent ischemic deficits. Cerebrovasc. Dis. 2006, 22:143-9.
8. Sun L. L., P. Ruff, B. Austin, S. Deb, B. Martin, D. Burris, et al. Early up-regulation of intercellular adhesion molecule-1 and vascular cell adhesion molecule-1 expression in rats with hemorrhagic shock and resuscitation. Shock 1999, 11:416-22.
9. Ferguson K. L., P. Taheri, J. Rodriguez, V. Tonapi, A. Cardellio, and R. Dechert. Tumor necrosis factor activity increases in the early response to trauma. Acad. Emerg. Med. 1997, 4(11):1035-40.
10. Mainous M. R., W. Ertel, I. H. Chaudry, and E. A. Deitch. The gut: a cytokine-generating organ in systemic inflammation? Shock 1995, 4:193-9.
11. Yang R., X. Han, T. Uchiyama, S. K. Watkins, A. Yaguchi, R. L. Delude, and M. P. Fink. IL-6 is essential for development of gut barrier dysfunction after hemorrhagic shock and resuscitation in mice. Am. J. Physiol. Gastrointest. Liver Physiol. 2003, 285(3):G621-9.
12. Taniguchi T., Y. Koido, J. Aiboshi, T. Yamashita, S. Suzaki, and A. Kurokawa. Change in the ratio of interleukin-6 to interleukin-10 predicts a poor outcome in patients with systemic inflammatory response syndrome. Crit. Care Med. 1999, 27:1262-1264.
13. Snyder E. L. The role of cytokines and adhesive molecules in febrile non-hemolytic transfusion reactions. Immunol. Invest. 1995, 24:333-9.
14. Winkelstein A., and J. E. Kiss. Immunohematologic disorders. Jama. 1997, 278:1982-92.
15. Davenport R. Cytokines and erythrocyte incompatibility. Curr. Opin. Hematol. 1994, 1:452-6.
16. Davenport R. D. The role of cytokines in hemolytic transfusion reactions. Immunol. Invest. 1995, 24:319-31.
17. Zhu X. L., R. Zellweger, X. H. Zhu, A. Ayala, and I. H. Chaudry. Cytokine gene expression in splenic macrophages and Kupffer cells following hemorrhage. Cytokine 1995, 7(1):8-14.
18. Fong Y., K. J. Tracey, L. L. Moldawer, D. G. Hesse, K. B. Manogue, J. S. Kenney, A. T.
Lee, G. C. Kuo, A. C. Allison, S. F. Lowry, and C. A. Cermani. Antibodies to cachectin/tumor necrosis factor reduce interleukin 1 beta and interleukin 6 appearance during lethal bacteremia. J. Exp. Med. 1989, 1; 170(5):1627-33.
19. Robinson M. K., J. D. Rounds, R. W. Hong, D. O. Jacobs, and D. W. Wilmore. Glutathione deficiency increases organ dysfunction after hemorrhagic shock. Surgery 1992, 112:140-7; discussion 8-9.
20. Sacks G., I. Sargent, and C. Redman. An innate view of human pregnancy. Immunol. Today 1999, 20(3):114-8.
21. Muyan M. and I. Boime I. Secretion of chorionic gondatrophin from human trophoblast. Placenta 1997, 19(4):237-41.
22. Alexander H., G. Zimmermann, M. Lehmann, R. Pfeiffer, E. Schone, S. Leiblein, and M. Ziegert. HCG secretion by peripheral mononuclear cells during pregnancy. Domest. Anim. Endocrinol. 1998, 15(5):377-87.
23. Khan N. A., A. Khan, H. F. Savelkoul, and R. Benner. Inhibition of diabetes in NOD mice by human pregnancy factor. Hum. Immunol. 2001, 62(12):1315-23.
24. Cole L. A. The deactivation of hCG by nicking and dissociation. J. Clin. Endocrinical. Metab. 1993, 76(3)704-10.
25. Benner R. and N. A. Khan. Dissection of systems, cell populations and molecules. Scand. J. Immunol. 2005, 62 Suppl 1:62-6.
26. Khan N. A., A. Khan, H. F. Savelkoul, and R. Benner. Inhibition of septic shock in mice by an oligopeptide from the beta-chain of human chorionic gonadotrophin hormone. Hum. Immunol. 2002, 63:1-7.
27. Beillard E., N. Pallisgaard, V. H. van der Velden, W. Bi, R. Dee, E. van der Schoot, E. Delabesse, E. Macintyre, E. Gottardi, G. Saglio, F. Watzinger, T. Lion, J. J. van Dongen, P. Hokland, and J. Gabert. Evaluation of candidate control genes for diagnosis and residual disease detection in leukemic patients using “real-time” quantitative reverse-transcriptase polymerase chain reaction (RQ-PCR)—a Europe against cancer program. Leukemia 2003, 17:2474-2486.
28. Childs E. W., J. G. Wood, D. M. Smalley, F. A. Hunter, and L. Y. Cheung. Leukocyte adherence and sequestration following hemorrhagic shock and total ischemia in rats. Shock 1999, 11(4):248-52.
29. Suenaert P., V. Bulteel, L. Lemmens, M. Noman, B. Geypens, G. Van Assche, K.
Geboes, J. L. Ceuppens, and P. Rutgeerts. Anti-tumor necrosis factor treatment restores the gut barrier in Crohn's disease. Am. J. Gastroenterol. 2002, 97(8):2000-4.
30. Olsen N. J. and C. M. Stein. New drugs for rheumatoid arthritis. N. Engl. J. Med. 2004, 350(21):2167-79.
31. Kox W. J., T. Volk, S. N. Kox, and H. D. Volk. Immunomodulatory therapies in sepsis. Intensive Care Med. 2000, 26 (1)5124-8.
32. Jit M., B. Henderson, M. Stevens, and R. M. Seymour. TNF-alpha neutralization in cytokine-driven diseases: a mathematical model to account for therapeutic success in rheumatoid arthritis but therapeutic failure in systemic inflammatory response syndrome. Rheumatology 2005 March, 44(3):323-31.
33. Yang R., X. Han, T. Uchiyama, S. K. Watkins, A. Yaguchi, R. L. Delude, and M. P. Fink. IL-6 is essential for development of gut barrier dysfunction after hemorrhagic shock and resuscitation in mice. Am. J. Physiol. Gastrointest. Liver Physiol. 2003, 285(3):G621-9.
34. Taniguchi T., Y. Koido, J. Aiboshi, T. Yamashita, S. Suzaki, and A. Kurokawa. Change in the ratio of interleukin-6 to interleukin-10 predicts a poor outcome in patients with systemic inflammatory response syndrome. Crit. Care Med. 1999, 27:1262-1264.
35. Stites D. P., C. S. Pavia, L. E. Clemens, R. W. Kuhn, and P. K. Siiteri. Immunologic regulation in pregnancy. Arthritis Rheum. 1979, 22(11):1300-7.
36. Saito S. Cytokine network at the feto-maternal interface. J. Reprod. Immunol. 2000, 47(2):87-103.
37. Puett D., Y. Li, G. DeMars, K. Angelova, and F. Fanelli. A functional transmembrane complex: the luteinizing hormone receptor with bound ligand and G protein. Mol. Cell. Endocrinol. 2007, 2, 260-262:126-36.
38. Bai J. P. F., P. Subramanian, H. I. Mosberg, and G. L. Amidon. Structural requirements for the intestinal mucosal-cell peptide transport: the need for N-terminal α-amino group. Pharm. Res. 1991, 8:593-599.
39. Yang R., X. Tan, A. M. Thomas, R. Steppacher, N. Qureshi, D. C. Morrison, and C. W. Van Way 3rd. Alanine-glutamine dipeptide (AGD) inhibits expression of inflammation-related genes in hemorrhagic shock. J. Parenter. Enteral. Nutr. 2007, 31(1):32-6.

### FURTHER REFERENCES

WO 99/59671,

WO 01/72831,

WO 97/49721,

WO 01/10907, and

WO 01/11048.

The contents of entirety of each of the references cited herein is incorporated in their entireties by this reference.

What is claimed is:

1. A method for modulating an iatrogenic event in a subject, the method comprising:
providing the subject with a peptide selected from the group consisting of LQG, AQG, LQGV (SEQ ID NO:1), AQGV (SEQ ID NO:2), LQGA (SEQ ID NO:3), VLPALP (SEQ ID NO:4), ALPALP (SEQ ID NO:5), VAPALP (SEQ ID NO:6), ALPALPQ (SEQ ID NO:7), VLPAAPQ (SEQ ID NO:8), VLPALAQ (SEQ ID NO:9), LAGV (SEQ ID NO:10), VLAALP (SEQ ID NO:11), VLPALA (SEQ ID NO:12), VLPALPQ (SEQ ID NO:13), VLAALPQ (SEQ ID NO:14), VLPALPA (SEQ ID NO:15), GVLPALP (SEQ ID NO:16), LPGC (SEQ ID NO:19), MTRV (SEQ ID NO:20), MTR, and VVC.

2. The method according to claim 1, wherein the peptide is selected from the group of LAGV (SEQ ID NO:10), AQGV (SEQ ID NO:2), VLPALPQ (SEQ ID NO:13), and LQGV (SEQ ID NO:1).

3. The method according to claim 1, wherein the peptide is selected from the group of LAGV (SEQ ID NO:10), AQGV (SEQ ID NO:2), and LQGV (SEQ ID NO:1).

4. The method according to any one of claims 1 to 3, wherein the subject is at risk of experiencing a systemic inflammatory response syndrome occurring after the event.

5. The method according to any one of claims 1 to 4, wherein the iatrogenic event comprises destruction or lysis of a cell or tissue of the subject or of a pathogen hosted by the subject.

6. The method according to claim 5, wherein the lysis is due to treatment of the subject with a pharmaceutical composition.


SEQUENCE LISTING (29 sequences)

SEQ ID NO: 1 - protein, 4 aa, Artificial Sequence
  Description of Artificial Sequence Synthetic peptide
LQGV

SEQ ID NO: 2 - protein, 4 aa, Artificial Sequence
  Description of Artificial Sequence Synthetic peptide
AQGV

SEQ ID NO: 3 - protein, 4 aa, Artificial Sequence
  Description of Artificial Sequence Synthetic peptide
LQGA

SEQ ID NO: 4 - protein, 6 aa, Artificial Sequence
  Description of Artificial Sequence Synthetic peptide
VLPALP

SEQ ID NO: 5 - protein, 6 aa, Artificial Sequence
  Description of Artificial Sequence Synthetic peptide
ALPALP

SEQ ID NO: 6 - protein, 6 aa, Artificial Sequence
  Description of Artificial Sequence Synthetic peptide
VAPALP

SEQ ID NO: 7 - protein, 7 aa, Artificial Sequence
  Description of Artificial Sequence Synthetic peptide
ALPALPQ

SEQ ID NO: 8 - protein, 7 aa, Artificial Sequence
  Description of Artificial Sequence Synthetic peptide
VLPAAPQ

SEQ ID NO: 9 - protein, 7 aa, Artificial Sequence
  Description of Artificial Sequence Synthetic peptide
VLPALAQ

SEQ ID NO: 10 - protein, 4 aa, Artificial Sequence
  Description of Artificial Sequence Synthetic peptide
LAGV

SEQ ID NO: 11 - protein, 6 aa, Artificial Sequence
  Description of Artificial Sequence Synthetic peptide
VLAALP

SEQ ID NO: 12 - protein, 6 aa, Artificial Sequence
  Description of Artificial Sequence Synthetic peptide
VLPALA

SEQ ID NO: 13 - protein, 7 aa, Artificial Sequence
  Description of Artificial Sequence Synthetic peptide
VLPALPQ

SEQ ID NO: 14 - protein, 7 aa, Artificial Sequence
  Description of Artificial Sequence Synthetic peptide
VLAALPQ

SEQ ID NO: 15 - protein, 7 aa, Artificial Sequence
  Description of Artificial Sequence Synthetic peptide
VLPALPA

SEQ ID NO: 16 - protein, 7 aa, Artificial Sequence
  Description of Artificial Sequence Synthetic peptide
GVLPALP

SEQ ID NO: 17 - protein, 13 aa, Artificial Sequence
  Description of Artificial Sequence Synthetic peptide
LQGVLPALPQVVC

SEQ ID NO: 18 - protein, 13 aa, Artificial Sequence
  Description of Artificial Sequence Synthetic peptide
LPGCPRGVNPVVS

SEQ ID NO: 19 - protein, 4 aa, Artificial Sequence
  Description of Artificial Sequence Synthetic peptide
LPGC

SEQ ID NO: 20 - protein, 4 aa, Artificial Sequence
  Description of Artificial Sequence Synthetic peptide
MTRV

SEQ ID NO: 21 - protein, 10 aa, Artificial Sequence
  Description of Artificial Sequence Synthetic peptide
VLPALPQVVC

SEQ ID NO: 22 - protein, 10 aa, Artificial Sequence
  Description of Artificial Sequence Synthetic peptide
LQGVLPALPQ

SEQ ID NO: 23 - protein, 8 aa, Artificial Sequence
  Description of Artificial Sequence Synthetic peptide
GVLPALPQ

SEQ ID NO: 24 - protein, 38 aa, Artificial Sequence
  Description of Artificial Sequence Synthetic peptide
VVCNYRDVRFESIRLPGCPRGVNPVVSYAVALSCQCAL

SEQ ID NO: 25 - protein, 35 aa, Artificial Sequence
  Description of Artificial Sequence Synthetic peptide
RPRCRPINATLAVEKEGCPVCITVNTTICAGYCPT

SEQ ID NO: 26 - protein, 18 aa, Artificial Sequence
  Description of Artificial Sequence Synthetic peptide
SKAPPPSLPSPSRLPGPS

SEQ ID NO: 27 - protein, 16 aa, Artificial Sequence
  Description of Artificial Sequence Synthetic peptide
SIRLPGCPRGVNPVVS

SEQ ID NO: 28 - DNA, 25 bp, Artificial Sequence
  Description of Artificial Sequence Synthetic probe
agctcagagggggactttccgagag

SEQ ID NO: 29 - protein, 4 aa, Artificial Sequence
  Description of Artificial Sequence Synthetic peptide
QVVC